(19) United States
(12) Patent Application Publication
(10) Pub. No.: US 2026/0193648 A1
(43) Pub. Date: Jul. 9, 2026

(54) ANTISENSE OLIGOMER (ASO) AS AN INHIBITOR OF LIVER FIBROSIS

(71) Applicant: THE REGENTS OF THE UNIVERSITY OF CALIFORNIA, Oakland, CA (US)

(72) Inventors: Jerrold M. Olefsky, Solana Beach, CA (US); Roi Isaac, San Diego, CA (US)

(21) Appl. No.: 19/130,095

(22) PCT Filed: Nov. 16, 2023
(86) PCT No.: PCT/US2023/080114

§ 371 Date: May 14, 2025

Related U.S. Application Data
(60) Provisional application No. 63/425,935, filed on Nov. 16, 2022.

(51) Int. Cl. C12N 15/113; A61K 31/712; A61K 31/713; A61P 1/16

(52) U.S. Cl. CPC C12N 15/113; A61K 31/712; A61K 31/713; A61P 1/16; C12N 2310/11; C12N 2310/14; C12N 2310/315; C12N 2310/322

(57) ABSTRACT

A method to prevent, inhibit or treat liver disease or cholangitis in a mammal is provided comprising: administering to the mammal a composition comprising an effective amount of an antisense oligonucleotide specific for human Hippo pathway transcription factor, TEAD1.

### CROSS-REFERENCE TO RELATED APPLICATIONS

This application claims the benefit of the filing date of U.S. application No. 63/425,935, filed on Nov. 16, 2022, the disclosure of which is incorporated by reference herein.

### STATEMENT OF GOVERNMENT SUPPORT

This invention was made with government support under DK063491 and DK101396 awarded by the National Institutes of Health. The government has certain rights in the invention.

### BACKGROUND

Non-alcoholic fatty liver (NAFL), characterized by excessive fat accumulation in the liver with mild or no inflammation, is the most common chronic liver condition in Western populations [1]. An estimated about 25% of patients with NAFL will develop Non-alcoholic steatohepatitis (NASH) characterized by steatosis, liver inflammation, hepatocyte damage (ballooning), and fibrosis [2]. NASH can also lead to cirrhosis, hepatocellular carcinoma, and eventually increased liver-related mortality [3].

Hepatic fibrosis is a primary predictor of mortality and adverse liver events in NASH patients and is caused by activation of hepatic stellate cells (HSCs) [4]. Thus, in NASH, quiescent HSCs undergo activation and can become proliferative, fibrogenic, contractile myofibroblasts, depositing a fibrous extracellular matrix (ECM), leading to liver scarring [4]. A complex signaling network among inflammation, cellular stress, exosomes, ECM interactions, and free cholesterol [5, 6] promotes the activation of HSCs. Thus, inhibiting activated HSCs is pivotal to the prevention and reduction of fibrosis in NASH [4]. The Hippo pathway regulates cell cycle progression, proliferation, apoptosis, and differentiation. Excessive activation of the Hippo pathway is associated with various human diseases, including inflammation, fibrosis, and cancers [7]. The transcriptional coactivator with PDZ-binding motif (TAZ) and Yes-associated protein (YAP) are two main effectors of the Hippo pathway [8]. The Hippo pathway kinases LATS1/2 phosphorylate YAP/TAZ which then associate with 14-3-3 proteins leading to sequestration in the cytoplasm. When the upstream Hippo pathway kinases are inactive, unphosphorylated YAP/TAZ translocate to the nucleus, interacting with the Hippo pathway transcription factor TEAD1-4 [9], promoting target gene expression. Notably, nuclear YAP/TAZ accumulation has been observed in activated HSCs [10, 11], and blocking the interaction between YAP/TAZ and TEAD, with the pharmacological inhibitor verteporfin results in inhibition of myofibroblast differentiation from HSCs [12].

The seven-transmembrane superfamily member three protein (TM7SF3) is a nuclear protein that can attenuate the development of ER stress and the unfolded protein response [13]. TM7SF3 forms complexes with a number of RNA-binding proteins/splicing factors such as HNRNPU, HNRNPK, and RBM14. Hence, TM7SF3 regulates alternative splicing under basal and stress conditions [14]. Unlike most seven-transmembrane proteins, TM7SF3 is ubiquitously expressed, including in the liver [15].

### SUMMARY

To assess the role of TM7SF3 in HSC activation, HSCs from TM7SF3 knockout mice as well as human HSCs were employed. It was found that the deletion of TM7SF3 in HSCs promotes activation and fibrosis in primary cultures and in liver organoids. Moreover, it was shown that TM7SF3 controls HSC activation by regulating critical components within the Hippo pathway, including TAZ and TEAD1. TM7SF3 inhibits TEAD1 alternative splicing promoting expression of the inactive form of TEAD1. Finally, antisense oligomers (ASOs) targeting TEAD1 pre-mRNA prevent alternative splicing into the active form of TEAD1. This inhibits the fibrogenic program in HSCs in vitro and mitigates the development of NASH in vivo.

Based on its inhibitory effect on hepatic stellate cells and fibrosis in the NASH mice model in vivo, ASO 56 could be used to treat other Liver-diseases that involve fibrosis, such as Primary Sclerosing Cholangitis (PSC). Furthermore, ASO 56 effectively modulates TEAD1 alternative splicing and promotes expression of the less active TEAD1 spliced variant in human fetal lung fibroblasts, (MRC5) and in normal adult lung fibroblasts (NALFs). These findings suggest that ASO 56 may be an effective treatment for a range of diseases that involve fibrosis.

In one embodiment, the disclosure provides a method to prevent, inhibit or treat liver disease or cholangitis in a mammal, comprising: administering to the mammal a composition comprising an effective amount of an isolated nucleic acid such as an antisense oligonucleotide specific for human Hippo pathway transcription factor, TEAD1. In one embodiment, the mammal is a human. In one embodiment, the composition is systemically administered. In one embodiment, the composition is orally administered. In one embodiment, the composition is injected. In one embodiment, an antisense oligonucleotide has at least 80%, 82%, 84%, 85%, 87%, 90%, 92%, 94%, 95%, 97%, 98% or 99% nucleotide sequence identity to ASO 56 (which has modified ribonucleotides relative to 73:1) or a portion thereof, e.g., the portion has 1, 2, 3 4, 5 or 6 fewer nucleotides at the 5′ and/or 3′ end relative to SEQ ID NO:1, with the activity of ASO 56. In one embodiment, an oligonucleotide has a length of about 20 to about 200 nucleotides, e.g., about 30 to about 150, about 50 to about 125, or about 75 to about 100 nucleotides. The nucleotides may be naturally occurring or may be modified. In one embodiment, the antisense oligonucleotide has at least one or more nucleotide analogs. In one embodiment, the mammal has or is at risk of having liver fibrosis. In one embodiment, the mammal has or is at risk of having Primary Sclerosing Cholangitis (PSC) or Non-alcoholic steatohepatitis (NASH).

Also provided is a method to prevent, inhibit or treat fibrosis in a mammal, comprising: administering to the mammal a composition comprising an effective amount of an isolated nucleic acid, such as an antisense oligonucleotide, specific for human Hippo pathway transcription factor, TEAD1. In one embodiment, the mammal is a human. In one embodiment, the composition is systemically administered. In one embodiment, the composition is orally administered. In one embodiment, the composition is injected. In one embodiment, the antisense oligonucleotide has at least 80%, 82%, 84%, 85%, 87%, 90%, 92%, 94%, 95%, 97%, 98% or 99% nucleotide sequence identity to ASO 56 or a portion thereof with the activity of ASO 56. In one embodiment, an oligonucleotide has a length of about 20 to about 200 nucleotides, e.g., about 30 to about 150, about 50 to about 125, or about 75 to about 100 nucleotides. The nucleotides may be naturally occurring or may be modified. In one embodiment, the antisense oligonucleotide has at least one or more nucleotide analogs. In one embodiment, the mammal has or is at risk of having liver fibrosis. In one embodiment, the composition comprises liposomes. In one embodiment, the composition comprises nanoparticles.

Further provided is a composition comprising an antisense oligonucleotide specific for human Hippo pathway transcription factor, TEAD1, e.g., an antisense oligonucleotide has at least 80%, 82%, 84%, 85%, 87%, 90%, 92%, 94%, 95%, 97%, 98% or 99% nucleotide sequence identity to ASO 56 (or or a portion thereof with the activity of ASO 56. In one embodiment, an oligonucleotide has a length of about 20 to about 200 nucleotides, e.g., about 30 to about 150, about 50 to about 125, or about 75 to about 100 nucleotides. The nucleotides may be naturally occurring or may be modified. In one embodiment, the composition comprises liposomes. In one embodiment, the composition comprises nanoparticles. In one embodiment, the composition comprises a sustained release dosage form.

### BRIEF DESCRIPTION OF THE FIGURES

FIGS. 1A-1I. TM7SF3 KO in HSCs induces fibrogenesis in liver organoids by promoting activation and cell proliferation. (FIGS. 1A-1E) WT hepatocytes and NPCs were assembled to form liver organoids, together with either WT or TM7SF3 KO HSCs as indicated (FIG. 1A). The liver organoids were then treated with either a high (FIG. 1B) or Low (FIG. 1C) dose of a NASH-inducing cocktail for 14 days and gene expression (FIGS. 1B-1C) protein (FIG. 1D), and secreted proteins were measured for the indicated genes. (FIG. 1F) Primary human HSCs were transfected with siRNA against TM7SF3, and gene expression was evaluated by qPCR. (FIGS. 1G-1H) Ki67 mRNA levels were measured in cultured mouse WT and KO HSCs (FIG. 1G) and KD human HSCs (FIG. 1H, as described in FIG. 1F). (FIG. 1I) In the same condition as IF, human HSCs were immuno-stained for ki67 protein ((Green) and DAPI (blue); the bar graph shows the quantitation of 2 experiments). Data are represented as mean+SEM (Panels FIGS. 1B, 1C, 1E-1H of 3-4 experiments; 1D and 1I—of 2 experiments in triplicates). See also FIG. 7.

FIGS. 2A-2L. TM7SF3 regulates HSC activation by modulation of TEAD1 alternative splicing. (FIG. 2A) RNA-seq of human liver samples, as indicated, shows TEAD1 exon 5 exclusion and expression of TEAD1^{ΔEx5}. (FIGS. 2B-2C) PCR of TEAD1 splicing in U2-OS and human HSCs transfected with siTM7SF3 and quantification (FIG. 2C). (FIG. 2D) A customized siRNA was designed to silence only the active form of TEAD1. (FIG. 2E) PCR of TEAD1 splicing in human HSCs transfected with the indicated siRNAs. (FIG. 2F) Quantitation of PCR products from IE. (FIG. 2G-2I). Gene expression with siRNA silencing of TM7SF3 with or without co-silencing of the active form of TEAD1 (siTEAD1^{ΔEx5}) in human HSCs. The cells were then treated with TGFβ for 24 hours. mRNA relative expression of Cyr61 (FIG. 2G), αSMA (FIG. 2H), and IL-6 (FIG. 2I) were detected by qPCR analysis. (FIG. 2J-2K) Mouse liver organoids were transfected with siTEAD1^{ΔEx5}, and fibrogenic markers were detected by qPCR (FIG. 2J) and ELISA (FIG. 2K). (FIG. 2L) Cell proliferation in the same experiment as described in 1G. Data are represented as mean+SEM of 3-4 experiments (Panel 2K-2 experiment in triplicates).

FIGS. 3A-3K. TM7SF3 gene inhibits TEAD1 alternative splicing via the hnRNPU splicing factor. (FIG. 3A) A list of potential splicing factors with a binding motif on the introns 100 bp up or downstream of the TEAD1 spliced exon 5. (FIG. 3B) Primer design for RNA-Immunoprecipitation (RIP). (FIG. 3C-3E) RNA-IP in human HSCs (FIG. 3C) and HSCs transfected with siRNA-hnRNPU (FIG. 3D) or siRNA-TM7SF3 I with the indicated antibodies. After incubation with antibodies, RNA was eluted, and PCR was performed. (FIG. 3F-3H) TM7SF3 was silenced by siRNA with or without co-silencing of hnRNPU in human HSCs. The cells were then treated with TGFβ for 24 hours and PCR detected full length TEAD1 and TEAD1^{ΔEx5}. (FIG. 3F) mRNA expression of the indicated genes was detected by qPCR analysis (FIGS. 3G-3H). (FIG. 3I) Scheme of the suggested model by which TM7SF3 regulates TEAD1 alternative splicing. TM7SF3 associates with hnRNPU and inhibits its activity to promote TEAD1 Exon 5 skipping, a. When TM7SF3 is depleted, hnRNPU is no longer inhibited and TEAD1^{ΔEx5} is increased. (FIG. 3K) In the same conditions as FIG. 3F, cell proliferation by Ki67 FACS immunostaining. Data are represented as mean+SEM of 3-4 experiments. See also FIG. 8.

FIGS. 4A-4I. ASO targeting TEAD1 pre mRNA deactivates HSCs. (FIG. 4A) an antisense oligomer (ASO) was designed to bind TEAD1 pre-mRNA and block the binding of hnRNPU. (FIG. 4B) human HSCs were transfected with the ASO for 48 hours. The cells were then treated with TGFβ for 24 hours. mRNA expression of the indicated genes was detected by qPCR analysis. (FIGS. 4C-4E) Mouse Liver organoids were transfected with the designed ASO (ASO 56), and fibrogenic markers were detected by qPCR (FIG. 4C), cellular protein expression (FIG. 4D), and secreted proteins by ELISA (FIG. 4E). (FIG. 4F-4G) Mice were injected with a single IV dose (10 mg/Kg) of ASO 56, and 7 days later, hepatocytes, NPCs, and HSCs were isolated by collagenase perfusion. RNA was extracted from cells, and TEAD1^{ΔEx5} (FIG. 4F) and Cyr61 gene expression (FIG. 4G) measured by qPCR. (FIG. 4H-4I) Mouse HSCs (FIG. 4H) and NPCs (FIG. 41) were transfected with the ASO 56 for 48 hours. The cells were then treated with TGFβ for 24 hours. mRNA expression of the indicated genes was detected by qPCR analysis. Data represent mean+SEM of 3-4 experiments (FIGS. 4D and 4I of 2 experiments). FIGS. 4F-4G—n=5 for each group. See also FIG. 9.

FIGS. 5A-5M. ASO targeting TEAD1 pre-mRNA deactivates HSCs in vivo. (FIG. 5A) scheme for ASO treatment protocol of mice fed with a Western diet for six months. (FIG. 5B-5I) After two months of ASO IV injections, Livers were removed, and markers of fibrogenic genes were detected by qPCR and protein. (FIG. 5B) Gene expression of TAZ, CTGF and Birc6 measured by qPCR. (FIG. 5C) Western blot of CTGF (FIG. 5D) A bar graph of CTGF immunoblot densitometry, normalized to actin. (FIG. 5E) Gene expression of TNFalpha measured by qPCR. (FIG. 5F-5G) Western blot of IL-1β, PDGFRβ and αSMA. (FIG. 5H) Bar graphs of IL-10, PDGFRβ and αSMA immunoblot densitometry, normalized to actin. (FIG. 5I) Timp1 protein detection by ELISA. (FIG. 5J) Blood ALT levels. (FIG. 5K) p21 protein detected by Western blot (FIG. 5L) Liver sections were stained with Sirius red for fibrosis detection. (FIG. 5M) Liver collagen levels were measured by hydroxyproline assay. Data are represented as mean+SEM; NCD n=4; ASO CON and 56 n=8; in FIGS. 5B, 5H, 5J, 5K and 5M data represents a meta-analysis of 2 cohorts. See also FIG. 10.

FIGS. 6A-6O. TM7SF3 KD regulation of HSC activation. (FIG. 6A-6B) Human HSCs were transfected with siTM7SF3 or siRNA-Control (siCON) and treated with TGFβ for 24 hours. Phosphorylation of seine 89 on TAZ protein was detected by Western blot (FIG. 6A) and quantitated (FIG. 6B). (FIG. 6C) human HSCs were transfected with siTM7SF3 and immuno-stained for TAZ protein (Green) and DAPI (blue). (FIG. 6D-6F) TM7SF3 mRNA was silenced by siRNA with or without co-silencing TAZ in human HSCs. The cells were then treated with TGFβ for 24 hours. mRNA expression of Cyr61 (FIG. 6D), αSMA (FIG. 6E), and PPARγ (FIG. 6F). (FIG. 6G) In the same conditions, cell proliferation was measured by WST-1 assay. (FIG. 6H) Naive human HSCs were incubated for 24 hours with conditioned media (CM) collected from human HSCs transfected with siTM7SF3 and then treated with TGFβ for 24 hours. RNA was extracted, and the indicated gene expression was evaluated by qPCR. (FIG. 6I) CTGF mRNA expression in the indicated cell types. (FIG. 6J-6K) CTGF mRNA expression, and secreted CTGF was precipitated by heparin beads and detected by Western blot (FIG. 6L) its quantification (FIG. 6M). (FIG. 6N) Co-silencing of TM7SF3 and CTGF in human HSCs, αSMA mRNA expression, and (FIG. 6O) cell proliferation were detected by qPCR analysis and WST-1 assay, respectively. Data are represented as mean+SEM of 3-4 experiments. See also FIG. 11.

FIGS. 7A-7H. TM7SF3 KO promotes activation and fibrogenesis in HSCs. Related to FIG. 1. (FIG. 7A) After silencing the TM7SF3 gene in mouse HSCs, RNA was extracted, and the indicated genes were measured by qPCR. (FIGS. 7B-7C) Human HSCs were transfected with siTM7SF3 or siRNA-Control (siCON) and further treated with TGFβ for 24 hours. TM7SF3 (FIG. 7B) and fibrogenic (FIG. 7C) protein levels were detected by Western blot and quantified. (FIG. 7D) Mouse NPCs were transfected with siRNA against TM7SF3 and gene expression was evaluated by qPCR. (FIG. 7E) WT and TM7SF3KO male mice were fed with NASH diet for 4 weeks, HSCs were isolated and gene expression was evaluated by qPCR. (FIGS. 7F-7H) HSCs were isolated from NCD WT and TM7SF3KO male mice and cultured for 4 days followed by TGFβ treatment for 24 hours. Gene expression was evaluated by qPCR (FIG. 7F) and protein levels of fibrogenic markers were detected by Western blot and quantified (FIGS. 7G-H).

FIGS. 8A-8D. TM7SF3 inhibits Tead1 alternative splicing via the hnRNPU splicing factor. Related to FIG. 3. (FIG. 8A) A PCR results for RNA-IP in U2-OS cells. (FIG. 8B-8D) TM7SF3 or hnRNPU genes were silenced by siRNA in human HSCs. The cells were then treated with TGFβ for 24 hours. mRNA expression of the indicated genes was detected by qPCR analysis.

FIGS. 9A-9D. ASO targeting TEAD1 pre mRNA deactivates HSCs. Related to FIG. 4. (A and B) Mouse hepatocytes, NPCs, and HSCs were isolated by collagenase perfusion and RNA was extracted from cells. TEAD1-4 (FIG. 9A) and hnRNPU (FIG. 9B) gene expression was detected by qPCR. (FIG. 9C-9D) Mouse HSCs were transfected with the ASO for 48 hours. The cells were treated with TGFβ for 24 hours. mRNA expression of the indicated genes was detected by qPCR analysis. Data are represented as mean+SEM of (FIGS. 9A-B) n=5 mice for each cell type. (FIG. 9C-9D) of 2 experiments.

FIGS. 10A-10C. ASO targeting TEAD1 pre mRNA deactivates HSCs in vivo. Related to FIG. 5. (A-C) from the experiment described in FIG. 5A, body weight (FIG. 10A), Liver mass (FIG. 10B), and epididymal fat mass (FIG. 10C) were measured. Data are represented as mean+SEM of n=5 mice for each cell type.

FIGS. 11A-11E. TM7SF3 KD promotes activation in U2-OS and HSCs. Related to FIG. 6. (FIG. 11A) U2-OS cells were transfected with siRNA against TM7SF3 and immuno-stained for TAZ protein (Green) and DAPI (blue). (FIGS. 12B-12D) TM7SF3 was silenced by siRNA with or without co-silencing TAZ in human HSCs. The cells were then treated with TGFβ for 24 hours. mRNA expression of TM7SF3 (FIG. 12B), TAZ (FIG. 12C), and TGFβ (FIG. 12D) were detected by qPCR analysis. (FIG. 12E) CTGF secreted protein levels from mouse liver organoids experiment (FIG. 12F) as detected by ELISA.

FIGS. 12A-12D. ASO 56 reduces blood ALT/AST levels in Mdr2 KO mice. (FIG. 12A) scheme for ASO treatment protocol in Mdr2 KO mice. (FIGS. 12B-12D) Blood ALT and/or AST levels were measured two weeks after the start of treatment (FIG. 12B) and at the end of the experimental protocol (FIG. 12C-12D). Data represent mean+SEM n=4.

FIGS. 13A-13G. The ASO targeting TEAD1 pre-mRNA causes HSC deactivation and reduces liver fibrosis in vivo. Livers were removed, and markers of fibrogenic genes were detected by qPCR (FIGS. 13A-13B). (FIG. 13C) Western blots of PDGFR and aSMA and bar graphs of immunoblot densitometry normalized to HSP90 (FIGS. 13D-13E). (FIG. 13F) Liver collagen levels were measured by the hydroxyproline assay. (FIG. 13G) Liver sections were stained with Sirius red for fibrosis detection (left panel) and quantified (right panel). Data represent mean+SEM; n=3-4.

FIGS. 14A-14C. ASO 56 reduces blood ALT/AST levels in Mdr2 KO mice. (FIG. 14A) Body weight after 4 weeks of treatment with ASO. (FIGS. 14B-14C) Blood ALT and AST levels were measured at the end of the experimental protocol. Data represent mean+SEM.

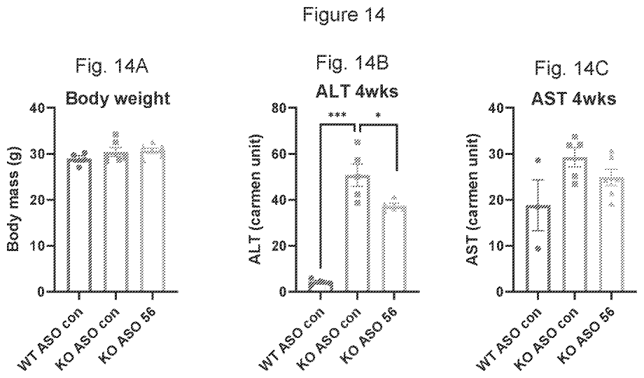

FIGS. 15A-15I. The ASO targeting TEAD1 pre-mRNA causes HSC deactivation and reduces liver fibrosis in vivo. Livers were removed, and markers of fibrogenic genes were detected by qPCR (FIG. 15A). (FIG. 15B) Liver Protein was extracted and run on SDS-PAGE under reducing conditions. Western blots of PDGFR and bar graphs of immunoblot densitometry normalized to HSP90 (FIG. 15C). (FIG. 15D) Liver Protein was extracted and run on SDS-PAGE under non-reducing conditions. Western blots of F4/80 and αSMA, and bar graphs of immunoblot densitometry, normalized to tubulin (FIGS. 15E-15F). (FIG. 15G) Liver collagen levels were measured by the hydroxyproline assay. (FIG. 15H) Liver sections were stained with Sirius red for fibrosis detection and quantified (FIG. 15I). Data represent mean+SEM.

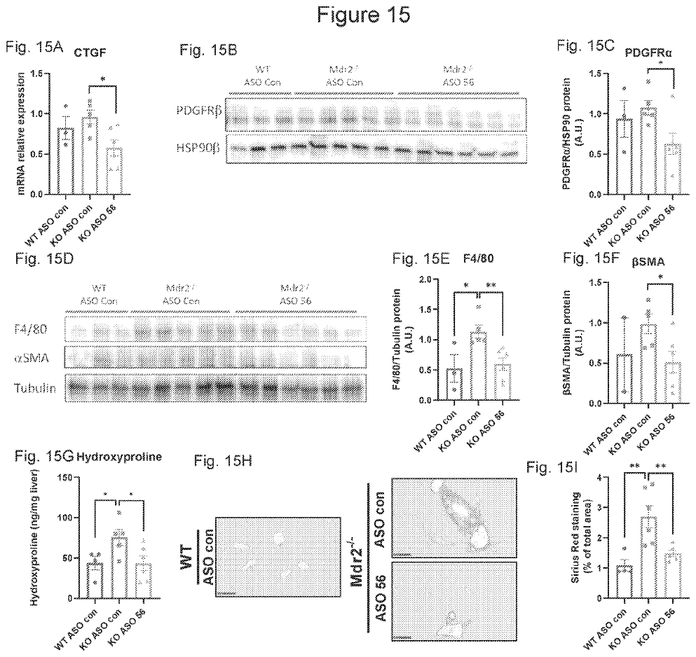

### DETAILED DESCRIPTION

The mechanisms of hepatic stellate cell (HSC) activation and the development of liver fibrosis are not fully understood. Here, we show that deletion of a nuclear seven transmembrane protein, TM7SF3, accelerates HSC activation in both mouse liver organoids and primary human HSCs, leading to activation of the fibrogenic program and proliferation. Thus, TM7SF3 inhibits the hnRNPU splicing factor, such that TM7SF3 KD promotes alternative splicing of the Hippo pathway transcription factor, TEAD1. This leads to exclusion of the inhibitory exon 5, generating an active form of TEAD1 that leads to HSC activation. Inhibition of TEAD1 alternative splicing with a specific ASO causes deactivation of HSCs in vilro and a reduction in liver fibrosis in vivo induced by a NASH diet. Finally, TM7SF3 KD promotes TAZ translocation to the nucleus. In conclusion, TM7SF3 modulates TEAD1 alternative splicing, inhibiting HSC activation and the development of NASH.

### Exemplary Non-Viral Delivery Vehicles for Isolated Nucleic Acid

In one embodiment, a non-viral delivery vehicle for nucleic acid comprises inorganic nanoparticles, e.g., calcium phosphate or silica particles; polymers including but not limited to poly(lactic-co-glycolic acid) (PLGA), polylactic acid (PLA), linear and/or branched PEI with differing molecular weights (e.g., 2, 22 and 25 kDa), dendrimers such as polyamidoamine (PAMAM) and polymethoacrylates; lipids including but not limited to cationic liposomes, cationic emulsions, DOTAP, DOTMA, DMRIE, DOSPA, distearoylphosphatidylcholine (DSPC), DOPE, or DC-cholesterol; peptide based vectors including but not limited to Poly-L-lysine or protamine; or poly(β-amino ester), chitosan, PEI-polyethylene glycol, PEI-mannose-dextrose, DOTAP-cholesterol or RNAiMAX.

In one embodiment, the delivery vehicle is a glycopolymer-based delivery vehicle, poly(glycoamidoamine)s (PGAAs), that have the ability to complex with various polynucleotide types and form nanoparticles. These materials are created by polymerizing the methylester or lactone derivatives of various carbohydrates (D-glucarate (D), meso-galactarate (G), D-mannarate (M), and L-tartarate (T)) with a series of oligoethyleneamine monomers (containing between 1-4 ethylenamines (Liu and Reineke, 2006). A subset composed of these carbohydrates and four ethyleneamines in the polymer repeat units yielded exceptional delivery efficiency.

In one embodiment, the delivery vehicle comprises polyethyleneimine (PEI), Polyamidoamine (PAMAM), PEI-PEG, PEI-PEG-mannose, dextran-PEI, OVA conjugate, PLGA microparticles, or PLGA microparticles coated with PAMAM.

In one embodiment, the delivery vehicle comprises a cationic lipid, e.g., N-[1-(2,3-dioleoyloxy)propel]-N,N,N-trimethylammonium (DOTMA), 2,3-dioleyloxy-N-[2-spermine carboxamide]ethyl-N,N-dimethyl-1-propanammonium trifluoracetate (DOSPA, Lipofectamine); 1,2-dioleoyl-3-trimethylammonium-propane (DOTAP); N-[1-(2,3-dimyristloxy) propyl]; N,N-dimethyl-N-(2-hydroxyethyl) ammonium bromide (DMRIE), 3-β-[N-(N,N-dimethylaminoethane) carbamoyl]cholesterol (DC-Chol); dioctadecyl amidoglyceryl spermine (DOGS, Transfectam); or imethyldioctadeclyammonium bromide (DDAB). The positively charged hydrophilic head group of cationic lipids usually consists of monoamine such as tertiary and quaternary amines, polyamine, amidinium, or guanidinium group. A series of pyridinium lipids have been developed (Zhu et al., 2008; van der Woude et al., 1997; Ilies et al., 2004). In addition to pyridinium cationic lipids, other types of heterocyclic head group include imidazole, piperizine and amino acid. The main function of cationic head groups is to condense negatively charged nucleic acids by means of electrostatic interaction to slightly positively charged nanoparticles, leading to enhanced cellular uptake and endosomal escape.

Lipids having two linear fatty acid chains, such as DOTMA, DOTAP and SAINT-2, or DODAC, may be employed as a delivery vehicle, as well as tetraalkyl lipid chain surfactant, the dimer of N,N-dioleyl-N,N-dimethylammonium chloride (DODAC). All the trans-orientated lipids regardless of their hydrophobic chain lengths (C_{16:1}, C_{18:1} and C_{20:1}) appear to enhance the transfection efficiency compared with their cis-orientated counterparts.

The structures of cationic polymers useful as a delivery vehicle include but are not limited to linear polymers such as chitosan and linear poly(ethyleneimine), branched polymers such as branch poly(ethyleneimine) (PEI), circle-like polymers such as cyclodextrin, network (crosslinked) type polymers such as crosslinked poly(amino acid) (PAA), and dendrimers. Dendrimers consist of a central core molecule, from which several highly branched arms ‘grow’ to form a tree-like structure with a manner of symmetry or asymmetry. Examples of dendrimers include polyamidoamine (PAMAM) and polypropylenimine (PPI) dendrimers.

DOPE and cholesterol are commonly used neutral co-lipids for preparing cationic liposomes. Branched PEI-cholesterol water-soluble lipopolymer conjugates self-assemble into cationic micelles. Pluronic (poloxamer), a non-ionic polymer and SP1017, which is the combination of Pluronics L61 and F127, may also be used.

In one embodiment, PLGA particles are employed to increase the encapsulation frequency although complex formation with PLL may also increase the encapsulation efficiency. Other cationic materials, for example, PEI, DOTMA, DC-Chol, or CTAB, may be used to make nanospheres.

In one embodiment, no delivery vehicle is employed, e.g., naked RNA is employed alone or with a scaffold.

In one embodiment, physical methods including but not limited to electroporation, sonoporation, magnetoporation, ultrasound or needle injection may be employed to introduce naked RNA, complexes of RNA and a delivery vehicle or RNA encapsulated in particles, or a scaffold having complexes of RNA and a delivery vehicle or RNA encapsulated in particles, into a tissue.

Numerous synthetic polymers may employed including polystyrene, poly-1-lactic acid (PLLA), polyglycolic acid (PGA) and poly-dl-lactic-co-glycolic acid (PLGA).

Biological materials such as collagen, various proteoglycans, alginate-based substrates and chitosan may be employed to deliver the nucleic acid. The natural polymers are also biodegradable and so allow host cells, over time, to produce their own extracellular matrix.

Collagen and collagen-GAG (CG) may be altered through physical and chemical cross-linking. Collagen-hydroxyapatite (CHA) and collagen-hydroxy apitite (CHA) may be employed. Suitable biocompatible materials for the polymers include but are not limited to polyacetic or polyglycolic acid and derivatives thereof, polyorthoesters, polyesters, polyurethanes, polyamino acids such as polylysine, lactic/glycolic acid copolymers, polyanhydrides and ion exchange resins such as sulfonated polytetrafluorethylene, polydimethyl siloxanes (silicone rubber) or combinations thereof.

In one embodiment, the polymer is formed from natural proteins or materials which may be crosslinked using a crosslinking agent such as 1-ethyl-3-(3-dimethylamino-propyl)carbodiimide hydrochloride. Such natural materials include albumin, collagen, fibrin, alginate, extracellular matrix (ECM), e.g., xenogeneic ECM, hyaluronan, chitosan, gelatin, keratin, potato starch hydrolyzed for use in electrophoresis, and agar-agar (agarose), or other “isolated materials”. An “isolated” material has been separated from at least one contaminant structure with which it is normally associated in its natural state such as in an organism or in an in vitro cultured cell population.

Other biocompatible materials include synthetic polymers in the form of hydrogels or other porous materials, e.g., permeable configurations or morphologies, such as polyvinyl alcohol, polyvinylpyrrolidone and polyacrylamide, polyethylene oxide, poly(2-hydroxyethyl methacrylate); natural polymers such as gums and starches; synthetic elastomers such as silicone rubber, polyurethane rubber; and natural rubbers, and include poly[α(4-aminobutyl)]-1-glycolic acid, polyethylene oxide (Roy et al., 2003), polyorthoesters (Heller et al., 2002), silk-elastin-like polymers (Megeld et al., 2002), alginate (Wee et al., 1998), EVAc (poly(ethylene-co-vinyl acetate), microspheres such as poly (D, L-lactide-co-glycolide) copolymer and poly (L-lactide), poly(N-isopropylacrylamide)-b-poly(D,L-lactide), a soy matrix such as one cross-linked with glyoxal and reinforced with a bioactive filler, e.g., hydroxylapatite, poly(epsilon-caprolactone)-poly(ethylene glycol) copolymers, poly(acryloyl hydroxyethyl) starch, polylysine-polyethylene glycol, an agarose hydrogel, or a lipid microtubule-hydrogel.

In one embodiment, complexes are embedded in or applied to a material including but not limited to hydrogels of poloxamers, polyacrylamide, poly(2-hydroxyethyl methacrylate), carboxyvinyl-polymers (e.g., Carbopol 934, Goodrich Chemical Co.), cellulose derivatives, e.g., methylcellulose, cellulose acetate and hydroxypropyl cellulose, polyvinyl pyrrolidone or polyvinyl alcohols, or combinations thereof.

In some embodiments, a biocompatible polymeric material is derived from a biodegradable polymeric such as collagen, e.g., hydroxylated collagen, fibrin, polylactic-polyglycolic acid, or a polyanhydride. Other examples include, without limitation, any biocompatible polymer, whether hydrophilic, hydrophobic, or amphiphilic, such as ethylene vinyl acetate copolymer (EVA), polymethyl methacrylate, polyamides, polycarbonates, polyesters, polyethylene, polypropylenes, polystyrenes, polyvinyl chloride, polytetrafluoroethylene, N-isopropylacrylamide copolymers, poly(ethylene oxide)/poly(propylene oxide) block copolymers, poly(ethylene glycol)/poly(D,L-lactide-co-glycolide) block copolymers, polyglycolide, polylactides (PLLA or PDLA), poly(caprolactone) (PCL), or poly(dioxanone) (PPS).

In another embodiment, the biocompatible material includes polyethyleneterephalate, polytetrafluoroethylene, copolymer of polyethylene oxide and polypropylene oxide, a combination of polyglycolic acid and polyhydroxyalkanoate, gelatin, alginate, poly-3-hydroxybutyrate, poly-4-hydroxybutyrate, and polyhydroxyoctanoate, and polyacrylonitrilepolyvinylchlorides.

In one embodiment, the following polymers may be employed, e.g., natural polymers such as starch, chitin, glycosaminoglycans, e.g., hyaluronic acid, dermatan sulfate and chrondrotin sulfate, and microbial polyesters, e.g., hydroxyalkanoates such as hydroxyvalerate and hydroxybutyrate copolymers, and synthetic polymers, e.g., poly(orthoesters) and polyanhydrides, and including homo and copolymers of glycolide and lactides (e.g., poly(L-lactide, poly(L-lactide-co-D,L-lactide), poly(L-lactide-co-glycolide, polyglycolide and poly(D,L-lactide), pol(D,L-lactide-coglycolide), poly(lactic acid colysine) and polycaprolactone.

In one embodiment, the biocompatible material for the distinct polymer is derived from isolated extracellular matrix (ECM). ECM may be isolated from endothelial layers of various cell populations, tissues and/or organs, e.g., any organ or tissue source including the dermis of the skin, liver, alimentary, respiratory, intestinal, urinary or genital tracks of a warm blooded vertebrate. ECM employed in the invention may be from a combination of sources. Isolated ECM may be prepared as a sheet, in particulate form, gel form and the like.

The biocompatible scaffold polymer may comprise silk, elastin, chitin, chitosan, poly(d-hydroxy acid), poly(anhydrides), or poly(orthoesters). More particularly, the biocompatible polymer may be formed polyethylene glycol, poly(lactic acid), poly(glycolic acid), copolymers of lactic and glycolic acid, copolymers of lactic and glycolic acid with polyethylene glycol, poly(E-caprolactone), poly(3-hydroxybutyrate), poly(p-dioxanone), polypropylene fumarate, poly(orthoesters), polyol/diketene acetals addition polymers, poly(sebacic anhydride) (PSA), poly(carboxybiscarboxyphenoxyphenoxy hexone (PCPP) poly[bis (p-carboxypheonoxy) methane](PCPM), copolymers of SA, CPP and CPM, poly(amino acids), poly(pseudo amino acids), polyphosphazenes, derivatives of poly[(dichloro)phosphazenes] or poly[(organo) phosphazenes], poly-hydroxybutyric acid, or S-caproic acid, polylactide-co-glycolide, polylactic acid, polyethylene glycol, cellulose, oxidized cellulose, alginate, gelatin or derivatives thereof.

Thus, the polymer employed as a scaffold may be formed of any of a wide range materials including polymers, including naturally occurring polymers, synthetic polymers, or a combination thereof. In one embodiment, the scaffold comprises biodegradable polymers. In one embodiment, a naturally occurring biodegradable polymer may be modified to provide for a synthetic biodegradable polymer derived from the naturally occurring polymer. In one embodiment, the polymer is a poly(lactic acid) (“PLA”) or poly(lactic-co-glycolic acid) (“PLGA”). In one embodiment, the scaffold polymer includes but is not limited to alginate, chitosan, poly(2-hydroxyethylmethacrylate), xyloglucan, co-polymers of 2-methacryloyloxyethyl phosphorylcholine, poly(vinyl alcohol), silicone, hydrophobic polyesters and hydrophilic polyester, poly(lactide-co-glycolide), N-isoproylacrylamide copolymers, poly(ethylene oxideypoly(propylene oxide), polylactic acid, poly(orthoesters), polyanhydrides, polyurethanes, copolymers of 2-hydroxyethylmethacrylate and sodium methacrylate, phosphorylcholine, cyclodextrins, polysulfone and polyvinylpyrrolidine, starch, poly-D,L-lactic acid-para-dioxanone-polyethylene glycol block copolymer, polypropylene, poly(ethylene terephthalate), poly(tetrafluoroethylene), poly-epsilon-caprolactone, or crosslinked chitosan hydrogels.

### Exemplary Lipids

In certain embodiments, one or more lipids in a delivery vehicle for isolated nucleic acid include one or more phosphatidyl-cholines (PCs) selected from 1,2-dimyristoyl-sn-glycero-3-phosphocholine (DMPC), 1,2-dioleoyl-3-trimethylammonium-propane (DOTAP), 1-palmitoyl-2-oleoyl-sn-glycero-3-phosphocholine (POPC), e.g., in a lipid mixture comprising between about 0.5% to about 20% or about 1% to about 10%, or about 5% to about 15%, of one or more unsaturated phosphatidyl-choline, DMPC [14:0] having a carbon length of 14 and no unsaturated bonds, 1,2-dipalmitoyl-sn-glycero-3-phosphocholine (DPPC) [16:0], 1,2-distearoyl-sn-glycero-3-phosphocholine (DSPC) [18:0], 1,2-dioleoyl-sn-glycero-3-phosphocholine (DOPC) [18:1 (Δ9-Cis)], POPC [16:0-18:1] or DOTAP [18:1]; cholesterol between about 10% to about 30% or about 25% to about 35%, or about 30% to about 40%; and/or a PEG modified lipid such as PEG-C-DMA (dimyristroylpropyl-3 amine) between about 1% to about 3%, about 1.2% to about 2.5%, or about 1.3% to about 2%.

In certain embodiments, the one or more lipids may include phospholipid, a phosphatidyl-choline, a phosphatidyl-serine, a phosphatidyl-diethanolamine, a phosphatidylinosite, a sphingolipid, or an ethoxylated sterol, or mixtures thereof. In illustrative examples of such embodiments, the phospholipid can be a lecithin; the phosphatidylinosite can be derived from soy, rape, cotton seed, egg and mixtures thereof; the sphingolipid can be ceramide, a cerebroside, a sphingosine, and a sphingomyelin, and a mixture thereof; the ethoxylated sterol can be phytosterol, PEG-(polyethyleneglycol)-5-soy bean sterol, and PEG-(polyethyleneglycol)-5 rapeseed sterol. In certain embodiments, the phytosterol comprises a mixture of at least two of the following compositions: sitosterol, campesterol and stigmasterol.

In still other illustrative embodiments, the one or more lipids are comprised of one or more phosphatidyl groups selected from the group consisting of phosphatidyl choline, phosphatidyl-ethanolamine, phosphatidyl-serine, phosphatidyl-inositol, lyso-phosphatidyl-choline, lyso-phosphatidyl-ethanolamnine, lyso-phosphatidyl-inositol and lyso-phosphatidyl-inositol.

In still other illustrative embodiments, the one or more lipids are comprised of phospholipid selected from a monoacyl or diacylphosphoglyceride.

In still other illustrative embodiments, the one or more lipids are comprised of one or more phosphoinositides selected from the group consisting of phosphatidyl-inositol-3-phosphate (PI-3-P), phosphatidyl-inositol-4-phosphate (PI-4-P), phosphatidyl-inositol-5-phosphate (PI-5-P), phosphatidyl-inositol-3,4-diphosphate (PI-3,4-P2), phosphatidyl-inositol-3,5-diphosphate (PI-3,5-P2), phosphatidyl-inositol-4,5-diphosphate (PI-4,5-P2), phosphatidyl-inositol-3,4,5-triphosphate (P1-3,4,5-P3), lysophosphatidyl-inositol-3-phosphate (LPI-3-P), lysophosphatidyl-inositol-4-phosphate (LPI-4-P), lysophosphatidyl-inositol-5-phosphate (LPI-5-P), lysophosphatidyl-inositol-3,4-diphosphate (LPI-3,4-P2), lysophosphatidyl-inositol-3,5-diphosphate (LPI-3,5-P2), lysophosphatidyl-inositol-4,5-diphosphate (LPI-4,5-P2), and lysophosphatidyl-inositol-3,4,5-triphosphate (LPI-3,4,5-P3), and phosphatidyl-inositol (PI), and lysophosphatidyl-inositol (LPI).

In still other illustrative embodiments, the one or more lipids are comprised of one or more phospholipids selected from the group consisting of PEG-poly(ethylene glycol)-derivatized distearoylphosphatidylethanolamine (PEG-DSPE), poly(ethylene glycol)-derivatized ceramides (PEG-CER), hydrogenated soy phosphatidylcholine (HSPC), egg phosphatidylcholine (EPC), phosphatidyl ethanolamine (PE), phosphatidyl glycerol (PG), phosphatidyl inositol (PI), monosialoganglioside, sphingomyelin (SPM), distearoylphosphatidylcholine (DSPC), dimyristoylphosphatidylcholine (DMPC), and dimyristoylphosphatidylglycerol (DMPG).

Other embodiments include the one or more lipids selected from 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine](DOPS), 1,2-dioleoyl-3-trimethylammonium-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy(polyethylene glycol)-2000](18:1 PEG-2000 PE), 1,2-dipalmitoyl-si-glycero-3-phosphoethanolamine-N-[methoxy(polyethylene glycol)-2000](16:0 PEG-2000 PE), 1-oleoyl-2-[12-[(7-nitro-2-1,3-benzoxadiazol-4-yl)amino]lauroyl]-sun-glycero-3-phosphocholine (18:1-12:0 NBD PC), 1-palmitoyl-2-(12-[(7-nitro-2-1,3-benzoxadiazol-4-yl)amino]lauroyl)-sn-glycero-3-phosphocholine (16:0-12:0 NBD PC), cholesterol and mixtures/combinations thereof, and wherein the lipid comprises a cationic lipid and optionally one or more zwitterionic phospholipids.

In one embodiment, lipids include, for example, 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine](DOPS), 1,2-dioleoyl-3-trimethylammonium-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy(polyethylene glycol)-2000](18:1 PEG-2000 PE), 1,2-dipalmitoyl-sn-glycero-3-phosphoethanolamine-N-[methoxy(polyethylene glycol)-2000](16:0 PEG-2000 PE), 1-Oleoyl-2-[12-[(7-nitro-2-1,3-benzoxadiazol-4-yl)amino]lauroyl]-sn-Glycero-3-Phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), cholesterol and mixtures/combinations thereof. Cholesterol, not technically a lipid, but presented as a lipid for purposes of an embodiment given the fact that cholesterol may be an important component of the lipid complexes.

Pegylated phospholipids maybe employed in the lipid complexes or nanoparticles, including for example, pegylated 1,2-distearoyl-sn-glycero-3-phosphoethanolamine (PEG-DSPE), pegylated 1,2-dioleoyl-sn-glycero-3-phosphoethanolamine (PEG-DOPE), pegylated 1,2-dipalmitoyl-sn-glycero-3-phosphoethanolamine (PEG-DPPE), PEG-C-DMA, and/or pegylated 1,2-dimyristoyl-sn-glycero-3-phosphoethanolamine (PEG-DMPE), among others, including a pegylated ceramide (e.g. N-octanoyl-sphingosine-1-succinylmethoxy-PEG or N-palmitoyl-sphingosine-1-succinylmethoxy-PEG, among others). The PEG generally ranges in size (average molecular weight for the PEG group) from about 350-7500, about 350-5000, about 500-2500, about 1000-2000. Pegylated phospholipids may comprise a portion of the lipid complexes or nanoparticles, e.g., they may comprise a minor component, or be absent. Accordingly, the percent by weight of a pegylated phospholipid in the complexes or nanoparticles ranges from 0% to 100% or 0.01% to 99%, e.g., about 0.5%, 1.0%, 1.5%, 2.0%, 2.5%, 3.0%, 3.5%, 4.0%, 5%, 10%, 15%, 20%, 25%, 30%, 35%, 40%, 50%, 55%, 60% and the remaining portion comprising at least one, two or three other lipid molecules, such as cholesterol, usually in amounts less than about 50% by weight, and one or more cationic lipids, usually in amounts less than about 60% by weight.

If the delivery vehicle comprises two or more distinct lipids, one of the lipids may be cationic, e.g., DOTAP, and at least one of the others is non-cationic, e.g., DPPC or DSPC. Ratios of the two or more distinct lipids can vary, for example, for two distinct lipids, the ratio of a non-cationic lipid, e.g., neutral lipid, to the cationic lipid may be 1:x wherein x>1, e.g., 2 or 2.5 or 3, or x=1.

The delivery vehicle may be formed from a single type of lipid, or a combination of two or more distinct lipids. For instance, one combination may include a cationic lipid and a neutral lipid, or a cationic lipid and a non-cationic lipid. Exemplary lipids for use in the cationic liposomes include but are not limited to DOTAP, DODAP, DDAB, DOTMA, MVL5, DPPC, DSPC, DOPE, DPOC, POPC, or any combination thereof. In one embodiment, the cationic liposome has one or more of the following lipids or precursors thereof:

Other lipids include N-[1-(2,3-dioleyloxy)propyl]-N,N,N-trimethylammonium chloride with a monovalent cationic head; N′,N′-dioctadecyl-N-4,8-diaza-10-aminodecanoyl glycine amide; 1,4,7,10-tetraazacyclododecane cyclen; imidazolium-containing cationic lipid having different hydrophobic regions (e.g., cholesterol and diosgenin); 1,2-dioleoyl-sn-glycero-3-phosphoethanolamine (DOPE); 3β-[N-(N,N-dimethylamino-ethane) carbamoyl) cholesterol (DC-Chol) and DOPE; 0,0′-ditetradecanoyl-N-(α-trimethyl ammonioacetyl) diethanol-amine chloride, DOPE and cholesterol, phosphatidylcholine; 1,2-dilinoleyl-4-(2-dimethylaminoethyl)-[1,3]-dioxolane, 1,2-distearoyl-sn-glycerol-3-phosphocholine (DSPC) and cholesterol, 1-palmitoyl-2-oleoyl-sn-glycero-3-phosphocholine, DOPE, and 1,2-distearoyl-sn-glycero-3-phosphoethanolamine-N-(methoxy[polyethylene glycol-2000), 1,2-di-0-octadecenyl-3-trimethylammonium propane, cholesterol, and D-α-toco; 1,2-dioleoyl-3-trimethylammonium-propane, cholesterol; 3-β(N-(N′,N′-dimethyl, N′-hydroxyethyl amino-propane) carbamoyl) cholesterol iodide, DMHAPC-Chol and DOPE in equimolar proportion, or 1-palmitoyl-2-oleoyl-sn-glycero-3-ethylphosphocholine:cholesterol, dimethyldioctadecylammonium (DDAB); 1,2-di-O-octadecenyl-3-trimethylammonium propane;N1-[2-((1S)-1-((3-aminopropyl)amino]-4-[di(3-amino-propyl)amino)amino]butylcarboxamido)ethyl]-3,4-di[oleyloxy]-benzamide (MVL5); 1,2-dioleoyl-3-dimethylammonium-propane (DODAP); 1,2-di-O-octadecenyl-3-trimethylammonium propane (DOTMA); 1,2-dioleoyl-sn-glycero-3-phosphocholine (DOPC); 1-palmitoyl-2-oleoyl-sn-glycero-3-phosphocholine (POPC).

### Pharmaceutical Compositions

The disclosure provides a composition comprising, consisting essentially of, or consisting of microparticles, nanoparticles, liposomes or lipid complexes comprising isolated nucleic acid, and optionally a pharmaceutically acceptable (e.g., physiologically acceptable) carrier. In one embodiment, additional components can be included that do not materially affect the composition (e.g., adjuvants, buffers, stabilizers, anti-inflammatory agents, solubilizers, preservatives, etc.). In one embodiment, when the composition consists of the nucleic acid and the delivery vehicle and optionally a scaffold or other pharmaceutically acceptable carrier, the composition does not comprise any additional components. Any suitable carrier can be used, and such carriers are well known in the art. The choice of carrier will be determined, in part, by the particular site to which the composition may be administered and the particular method used to administer the composition. The composition optionally can be sterile. The composition can be frozen or lyophilized for storage and reconstituted in a suitable sterile carrier prior to use. The compositions can be generated in accordance with conventional techniques described in, e.g., Remington: The Science and Practice of Pharmacy, 21st Edition, Lippincott Williams & Wilkins, Philadelphia, PA (2001).

Suitable formulations for the composition include aqueous and non-aqueous solutions, isotonic sterile solutions, which can contain anti-oxidants, buffers, and bacteriostats, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid carrier, for example, water, immediately prior to use. Extemporaneous solutions and suspensions can be prepared from sterile powders, granules, and tablets of the kind previously described. In one embodiment, the carrier is a buffered saline solution. In one embodiment, the therapeutic nucleic acid is administered in a composition formulated to protect the therapeutic nucleic acid from damage prior to administration. In addition, one of ordinary skill in the art will appreciate that the therapeutic nucleic acid can be present in a composition with other therapeutic or biologically-active agents.

Injectable depot forms are envisioned including those having biodegradable polymers such as polylactide-polyglycolide. Depending on the ratio of inhibitor to polymer, and the nature of the particular polymer employed, the rate of inhibitor release can be controlled. Examples of other biodegradable polymers include poly(orthoesters) and poly(anhydrides). Depot injectable formulations are also prepared by entrapping the nucleic acid optionally in a complex with a delivery vehicle in liposomes or other lipid complexes or microemulsions which are compatible with body tissue.

In certain embodiments, a formulation comprises a biocompatible polymer selected from the group consisting of polyamides, polycarbonates, polyalkylenes, polymers of acrylic and methacrylic esters, polyvinyl polymers, polyglycolides, polysiloxanes, polyurethanes and co-polymers thereof, celluloses, polypropylene, polyethylenes, polystyrene, polymers of lactic acid and glycolic acid, polyanhydrides, poly(ortho)esters, poly(butic acid), poly(valeric acid), poly(lactide-co-caprolactone), polysaccharides, proteins, polyhyaluronic acids, polycyanoacrylates, and blends, mixtures, or copolymers thereof.

The composition can be administered in or on a device that allows controlled or sustained release, such as a sponge, biocompatible meshwork, mechanical reservoir, or mechanical implant. Implants (see, e.g., U.S. Pat. No. 5,443,505), devices (see, e.g., U.S. Pat. No. 4,863,457), such as an implantable device, e.g., a mechanical reservoir or an implant or a device comprised of a polymeric composition, are particularly useful for administration. The composition also can be administered in the form of sustained-release formulations (see, e.g., U.S. Pat. No. 5,378,475) comprising, for example, gel foam, hyaluronic acid, gelatin, chondroitin sulfate, a polyphosphoester, such as bis-2-hydroxyethyl-terephthalate (BHET), and/or a polylactic-glycolic acid.

The dose of the nucleic acid in the composition administered to the mammal will depend on a number of factors, including the size (mass) of the mammal, the extent of any side-effects, the particular route of administration, and the like. In one embodiment, the method comprises administering a “therapeutically effective amount” of the composition. A “therapeutically effective amount” refers to an amount effective, at dosages and for periods of time necessary, to achieve a desired therapeutic result. The therapeutically effective amount may vary according to factors such as the extent of the disease or disorder, age, sex, and weight of the individual, and the ability of the protease inhibitor to elicit a desired response in the individual. One of ordinary skill in the art can readily determine an appropriate protease inhibitor dose range to treat a patient having a particular disease or disorder, based on these and other factors that are well known in the art.

In one embodiment, the composition is administered once to the mammal. It is believed that a single administration of the composition may result in persistent expression in the mammal, optionally with minimal side effects. However, in certain cases, it may be appropriate to administer the composition multiple times during a therapeutic period to ensure sufficient exposure of cells to the composition. For example, the composition may be administered to the mammal two or more times (e.g., 2, 3, 4, 5, 6, 6, 8, 9, or 10 or more times) during a therapeutic period.

The present disclosure provides pharmaceutically acceptable compositions which comprise a therapeutically-effective amount of the nucleic acid, optionally on a plasmid or viral vector, e.g., an AAV or lentivirus vector.

### Exemplary Formulations, Dosages and Routes of Administration

The isolated nucleic acid, e.g., DNA or RNA or modified forms thereof, for example, one having SEQ ID NO:1 or variants thereof having at least 90% nucleic acid identity thereto, can be formulated as pharmaceutical compositions and administered to a mammalian host, such as a human patient in a variety of forms adapted to the chosen route of administration, e.g., orally or parenterally, by intravenous (IV), intraperitoneal (IP), intramuscular, topical, local, or subcutaneous routes. In one embodiment, the composition having isolated polypeptide or peptide is administered to a site of bone loss or cartilage damage or is administered prophylactically.

In one embodiment, the isolated nucleic acid may be administered by infusion or injection. Solutions of the nucleic acid or its salts, can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion may include sterile aqueous solutions or dispersions or sterile powders comprising the active ingredient which are adapted for the extemporaneous preparation of sterile injectable or infusible solutions or dispersions, optionally encapsulated in complexes, liposomes, nanoparticles or microparticles. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In some cases, it may be desirable to include isotonic agents, for example, sugars, buffers or sodium chloride. Prolonged absorption of the injectable compositions can be brought about by the use in the compositions of agents delaying absorption, for example, microparticles, or aluminum monostearate and gelatin.

Sterile injectable solutions are prepared by incorporating the active agent in the required amount in the appropriate solvent with various of the other ingredients enumerated above, as required, followed by filter sterilization. In the case of sterile powders for the preparation of sterile injectable solutions, the methods of preparation include vacuum drying and the freeze-drying techniques, which yield a powder of the active ingredient plus any additional desired ingredient present in the previously sterile-filtered solutions.

Useful solid carriers may include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as antimicrobial agents can be added to optimize the properties for a given use. Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Useful dosages of the isolated nucleic acid can be determined by comparing their in vitro activity and in vitro activity in animal models thereof. Methods for the extrapolation of effective dosages in mice, and other animals, to humans are known to the art; for example, see U.S. Pat. No. 4,938,949.

Generally, the concentration of the isolated nucleic acid in a liquid composition, may be from about 0.1-25 wt-%, e.g., from about 0.5-10 wt-%. The concentration in a semi-solid or solid composition such as a gel or a powder may be about 0.1-5 wt-%, e.g., about 0.5-2.5 wt-%.

The amount of the isolated nucleic acid for use alone or with other agents will vary with the route of administration, the nature of the condition being treated and the age and condition of the patient and will be ultimately at the discretion of the attendant physician or clinician.

The isolated nucleic acid may be conveniently administered in unit dosage form; for example, containing 5 to 1000 mg, conveniently 10 to 750 mg, or conveniently 50 to 500 mg of active ingredient per unit dosage form.

In general, a suitable dose of nucleic acid may be in the range of from about 0.5 to about 100 mg/kg, e.g., from about 10 to about 75 mg/kg of body weight per day, such as 3 to about 50 mg per kilogram body weight of the recipient per day, for example in the range of 6 to 90 mg/kg/day, e.g., in the range of 15 to 60 mg/kg/day.

For viral vectors, the dose may be from about 1×10⁴ GC/kg, about 1×10⁵ GC/kg, about 1×10⁶ GC/kg, about 1×10⁷ GC/kg, about 1×10⁸ GC/kg, about 1×10⁹ GC/kg, about 1×10¹⁰ GC/kg, such as 1×10¹¹ GC/kg, 2×10¹¹ GC/kg, 3×10¹¹ GC/kg, 4×10¹¹ GC/kg, 5×10¹¹ GC/kg, 6×10¹¹ GC/kg, 7×10¹¹ GC/kg, 8×10¹¹ GC/kg, 9×10¹¹ GC/kg, or 1×10¹² GC/kg.

The ultimate dosage form may be sterile, fluid and stable under the conditions of manufacture and storage. The liquid carrier or vehicle may be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The prevention of the action of microorganisms can be brought about by various antibacterial and antifungal agents, for example, parabens, chlorobutanol, phenol, sorbic acid, thimerosal, and the like. In many cases, it may be desirable to include isotonic agents, for example, sugars, buffers or sodium chloride.

Both local administration and systemic administration are contemplated. One or more suitable unit dosage forms can be administered by a variety of routes including local. The formulations may, where appropriate, be conveniently presented in discrete unit dosage forms and may be prepared by any of the methods well known to pharmacy. Such methods may include the step of bringing into association the subunit components, e.g., one or more lipids, subunits of a polymer or co-polymer, or the polymer or co-polymer, and the RNA and optionally liquid carriers, solid matrices, semi-solid carriers, finely divided solid carriers or combinations thereof, and then, if necessary, introducing or shaping the product into the desired delivery system.

The delivery vehicle such as a pharmaceutically acceptable carrier(s) may conveniently be provided in the form of formulations suitable for administration. A suitable administration format may best be determined by a medical practitioner for each patient individually, according to standard procedures. Suitable pharmaceutically acceptable carriers and their formulation are described in standard formulations treatises, e.g., Remington's Pharmaceuticals Sciences. By “pharmaceutically acceptable” it is meant a carrier, diluent, excipient, and/or salt that is compatible with the other ingredients of the formulation, and not deleterious to the recipient thereof.

The active agent may be formulated in solution at neutral pH, for example, about pH 6.5 to about pH 8.5, or from about pH 7 to 8, with an excipient to bring the solution to about isotonicity, for example, 4.5% mannitol or 0.9% sodium chloride, pH buffered with art-known buffer solutions, such as sodium phosphate, that are generally regarded as safe, together with an accepted preservative such as metacresol 0.1% to 0.75%, or from 0.15% to 0.4% metacresol. Obtaining a desired isotonicity can be accomplished using sodium chloride or other pharmaceutically acceptable agents such as dextrose, boric acid, sodium tartrate, propylene glycol, polyols (such as mannitol and sorbitol), or other inorganic or organic solutes. Sodium chloride is useful for buffers containing sodium ions. If desired, solutions of the above compositions can also be prepared to enhance shelf life and stability. Therapeutically useful compositions can be prepared by mixing the ingredients following generally accepted procedures. For example, the selected components can be mixed to produce a concentrated mixture which may then be adjusted to the final concentration and viscosity by the addition of water and/or a buffer to control pH or an additional solute to control tonicity.

In one embodiment, the nucleic acid, e.g., DNA or RNA or modified forms thereof, may be formulated for administration, e.g., by injection, infusion, a pump or a catheter, and may be presented in unit dose form in ampules, pre-filled syringes, small volume infusion containers or in multi-dose containers with an added preservative. The active ingredients may take such forms as suspensions, solutions, or emulsions in oily or aqueous vehicles, and may contain formulary agents such as suspending, stabilizing and/or dispersing agents. Alternatively, the active ingredients may be in powder form, obtained by aseptic isolation of sterile solid or by lyophilization from solution, for constitution with a suitable vehicle, e.g., sterile, pyrogen-free water, before use.

The formulations and compositions described herein may also contain other ingredients such as antimicrobial agents or preservatives.

The pharmaceutical formulations can also take the form of an aqueous or anhydrous solution, e.g., a lyophilized formulation, or dispersion, or alternatively the form of an emulsion or suspension.

### Exemplary Routes of Administration, Dosages and Dosage Forms for ASOs

Administration of ASO, e.g., DNA or RNA or modified forms thereof, may be continuous or intermittent, depending, for example, upon the recipient's physiological condition, and other factors known to skilled practitioners. The administration of the therapeutic agent may be essentially continuous over a preselected period of time or may be in a series of spaced doses. Both local administration, e.g., to a site of a bone defect, and systemic administration are contemplated. Any route of administration may be employed, e.g., intravenous, intranasal or intrabronchial, or local administration. In one embodiment, compositions may be subcutaneously, orally or intravascularly delivered.

One or more suitable unit dosage forms comprising the ASO, which may optionally be formulated for sustained release, can be administered by a variety of routes including local, e.g., intrathecal, oral, or parenteral, including by rectal, buccal, vaginal and sublingual, transdermal, subcutaneous, intravenous, intramuscular, intraperitoneal, intrathoracic, or intrapulmonary routes. The formulations may, where appropriate, be conveniently presented in discrete unit dosage forms and may be prepared by any of the methods well known to pharmacy. Such methods may include the step of bringing into association the therapeutic nucleic acid with liquid carriers, solid matrices, semi-solid carriers, finely divided solid carriers or combinations thereof, and then, if necessary, introducing or shaping the product into the desired delivery system.

The amount of ASO administered to achieve a particular outcome will vary depending on various factors including, but not limited to the condition, patient specific parameters, e.g., height, weight and age, and whether prevention or treatment, is to be achieved.

The ASO may conveniently be provided in the form of formulations suitable for administration. A suitable administration format may best be determined by a medical practitioner for each patient individually, according to standard procedures. Suitable pharmaceutically acceptable carriers and their formulation are described in standard formulations treatises, e.g., Remington's Pharmaceuticals Sciences. By “pharmaceutically acceptable” it is meant a carrier, diluent, excipient, and/or salt that is compatible with the other ingredients of the formulation, and not deleterious to the recipient thereof.

The complexes or particles containing ASO molecules may be formulated in solution at neutral pH, for example, about pH 6.5 to about pH 8.5, or from about pH 7 to 8, with an excipient to bring the solution to about isotonicity, for example, 4.5% mannitol or 0.9% sodium chloride, pH buffered with art-known buffer solutions, such as sodium phosphate, that are generally regarded as safe, together with an accepted preservative such as metacresol 0.1% to 0.75%, or from 0.15% to 0.4% metacresol. Obtaining a desired isotonicity can be accomplished using sodium chloride or other pharmaceutically acceptable agents such as dextrose, boric acid, sodium tartrate, propylene glycol, polyols (such as mannitol and sorbitol), or other inorganic or organic solutes. Sodium chloride is useful for buffers containing sodium ions. If desired, solutions of the above compositions can also be prepared to enhance shelf life and stability. Therapeutically useful compositions can be prepared by mixing the ingredients following generally accepted procedures. For example, the selected components can be mixed to produce a concentrated mixture which may then be adjusted to the final concentration and viscosity by the addition of water and/or a buffer to control pH or an additional solute to control tonicity.

The ASO can be provided in a dosage form containing an amount effective in one or multiple doses. The therapeutic nucleic acid may be administered in dosages of at least about 0.0001 mg/kg to about 20 mg/kg, of at least about 0.001 mg/kg to about 0.5 mg/kg, at least about 0.01 mg/kg to about 0.25 mg/kg, at least about 0.1 mg/kg to about 0.25 mg/kg of body weight, about 0.1 mg/kg to about 0.5 mg/kg, about 0.5 mg/kg to about 2 mg/kg, about 1 mg/kg to about 5 mg/kg, about 5 mg/kg to about 10 mg/kg, or about 10 mg/kg to about 20 mg/kg although other dosages may provide beneficial results. The amount administered will vary depending on various factors including, but not limited to, the disease, the weight, the physical condition, the health, and/or the age of the mammal. Such factors can be readily determined by the clinician employing animal models or other test systems that are available in the art. As noted, the exact dose to be administered is determined by the attending clinician but may be in 1 mL phosphate buffered saline. In one embodiment, from 0.0001 to 1 mg or more, e.g., up to 1 g, in individual or divided doses, e.g., from 0.001 to 0.5 mg, or 0.01 to 0.1 mg, of therapeutic nucleic acid can be administered.

Pharmaceutical formulations containing the isolated ASO can be prepared by procedures known in the art using well known and readily available ingredients. For example, the agent can be formulated with common excipients, diluents, or carriers, and formed into tablets, capsules, suspensions, powders, and the like. The isolated ASO containing particles or complexes can also be formulated as elixirs or solutions appropriate for parenteral administration, for instance, by intramuscular, subcutaneous or intravenous routes.

The pharmaceutical formulations can also take the form of an aqueous or anhydrous solution, e.g., a lyophilized formulation, or dispersion, or alternatively the form of an emulsion or suspension.

In one embodiment, isolated ASO containing particles or complexes may be formulated for administration, e.g., by injection, for example, bolus injection or continuous infusion via a catheter, and may be presented in unit dose form in ampules, pre-filled syringes, small volume infusion containers or in multi-dose containers with an added preservative. The active ingredients may take such forms as suspensions, solutions, or emulsions in oily or aqueous vehicles, and may contain formulatory agents such as suspending, stabilizing and/or dispersing agents. Alternatively, the active ingredients may be in powder form, obtained by aseptic isolation of sterile solid or by lyophilization from solution, for constitution with a suitable vehicle, e.g., sterile, pyrogen-free water, before use.

These formulations can contain pharmaceutically acceptable vehicles and adjuvants which are well known in the prior art. It is possible, for example, to prepare solutions using one or more organic solvent(s) that is/are acceptable from the physiological standpoint.

For administration to the upper (nasal) or lower respiratory tract by inhalation, the ASO composition is conveniently delivered from an insufflator, nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount.

Alternatively, for administration by inhalation or insufflation, the composition may take the form of a dry powder, for example, a powder mix of the therapeutic agent and a suitable powder base such as lactose or starch. The powder composition may be presented in unit dosage form in, for example, capsules or cartridges, or, e.g., gelatine or blister packs from which the powder may be administered with the aid of an inhalator, insufflator or a metered-dose inhaler.

For intra-nasal administration, the ASO composition may be administered via nose drops, a liquid spray, such as via a plastic bottle atomizer or metered-dose inhaler. Typical of atomizers are the Mistometer (Wintrop) and the Medihaler (Riker).

The local delivery of the ASO composition can also be by a variety of techniques which administer the therapeutic nucleic acid composition at or near the site of disease, e.g., using a catheter or needle. Examples of site-specific or targeted local delivery techniques are not intended to be limiting but to be illustrative of the techniques available. Examples include local delivery catheters, such as an infusion or indwelling catheter, e.g., a needle infusion catheter, shunts and stents or other implantable devices, site specific carriers, direct injection, or direct applications.

The formulations and compositions described herein may also contain other ingredients such as antimicrobial agents or preservatives.

### Subjects

The subject may be any animal, including a human and non-human animals. Non-human animals include all vertebrates, e.g., mammals and non-mammals, such as non-human primates, sheep, dogs, cats, cows, horses, chickens, amphibians, and reptiles, although mammals, such as non-human primates, sheep, dogs, cats, cows and horses, are envisioned. The subject may also be livestock such as, cattle, swine, sheep, poultry, and horses, or pets, such as dogs and cats.

Subjects include human subjects suffering from or at risk for oxidative damage. The subject is generally diagnosed with the condition of the subject invention by skilled artisans, such as a medical practitioner.

The methods described herein can be employed for subjects of any species, gender, age, ethnic population, or genotype. Accordingly, the term subject includes males and females, and it includes elderly, elderly-to-adult transition age subjects adults, adult-to-pre-adult transition age subjects, and pre-adults, including adolescents, children, and infants.

Examples of human ethnic populations include Caucasians, Asians, Hispanics, Africans, African Americans, Native Americans, Semites, and Pacific Islanders. The methods of the invention may be more appropriate for some ethnic populations such as Caucasians, especially northern European populations, as well as Asian populations.

The term subject also includes subjects of any genotype or phenotype as long as they are in need of the invention, as described above. In addition, the subject can have the genotype or phenotype for any hair color, eye color, skin color or any combination thereof.

The term subject includes a subject of any body height, body weight, or any organ or body part size or shape.

### Exemplary Sequences for Delivery

The compositions and/or vectors may have or express DNA or RNA corresponding to SEQ ID NO:1 or a sequence with one or more modified nucleotides. Exemplary modified forms may include 2MOE-C, 2MOE-A, 2MOE-U, and/or MOE-G, or any combination thereof. Exemplary ASOs may include but are not limited to:

(SEQ ID NO: 74)
5′-XYZCUAAAAUACAGAAUACX1Y1Z1-3′

wherein X or Y1 independently is G, A or U or a modified form thereof;
wherein Y or Z1 independently is G, A or U or a modified form thereof;
wherein Z independently is C, A or U or a modified form thereof; or
wherein X1 independently is C, A or G or a modified form thereof;

(SEQ ID NO: 75)
5′-CAGX2Y2Z2AAAUACAGAAX3Y3Z3UCA-3′

wherein X2 or Z3 independently is G, A or U or a modified form thereof;
wherein Y2 or X3 independently is G, A or C or a modified form thereof;
or
wherein Z2 or Y3 independently is C, U or G;

(SEQ ID NO: 76)
5′-X4AGX4UAAAAUAX4AGAAUAX4UCA-3′

wherein one or more X4 independently is G, U or A or a modified form thereof.

(SEQ ID NO: 77)
5′-CX5GCUX5X5X5X5UX5CX5GX5UX5CUCX4-3′

wherein one or more X5 independently is G, U or C or a modified form thereof.

(SEQ ID NO: 78)
5′-CAX6CUAAAAUACAX6AAUACUCA-3′

wherein one or more X6 independently is C, U or A or a modified form thereof.

(SEQ ID NO: 79)
5′-CAGCX7AAAAX7ACAGAAX7ACX7CA-3′

wherein one or more X7 independently is G, C or A or a modified form thereof.

The invention will be further described by the following non-limiting examples.

### Example I

### Exemplary Materials and Methods

Antibodies

| | | |
|---|---|---|
| Rabbit polyclonal anti-HSP90 | Cell Signaling Technology | Cat#: 4874 |
| | | RRID: AB_2121214 |
| Rabbit polyclonal anti-Actin | Cell Signaling Technology | Cat# 4970 |
| | | RRID: AB_2223172 |
| Rabbit polyclonal anti-Ki67 | Abcam | Cat#: ab15580 |
| | | RRID: AB_443209 |
| Rabbit polyclonal anti-CTGF | Abcam | Cat#: ab6992 |
| | | RRID: AB_305688 |
| Rabbit polyclonal anti-WWTR1 | Sigma | Cat#hpa0077415 |
| Mouse monoclonal anti-Alpha-SMA | Invitrogen | Cat#14-9760-82 |
| | | RRID: AB_2572996 |
| Rabbit anti-IgG | Santa Cruz Biotechnology | Cat#: sc-2027 |
| | | RRID: AB_737197 |
| Mouse anti-IgG2B | Santa Cruz Biotechnology | Cat#: sc-3879 |
| | | RRID: AB_737262 |
| Mouse monoclonal anti-p21 | Santa Cruz Biotechnology | Cat#: sc-6246 |
| | | RRID: AB_628073 |
| Rabbit polyclonal anti-IL1-□ | Santa Cruz Biotechnology | Cat#: sc-7884 |
| | | RRID: AB_2124476 |
| Mouse monoclonal anti-hnRNPU | Santa Cruz Biotechnology | Cat#: sc-32315 |
| | | RRID: AB_627741 |
| Mouse monoclonal anti-Col1a1 | Santa Cruz Biotechnology | Cat#: sc-293182 |
| | | RRID: AB_2797597 |
| Rabbit polyclonal anti-pSer89-TAZ | Santa Cruz | Cat#: sc-17610-R |

| | |
|---|---|
| REAGENT or RESOURCE | |
| IDENTIFIER | SOURCE |

Antibodies

| | | |
|---|---|---|
| Rabbit polyclonal anti-HSP90 | Cell Signaling | Cat#: 4874 |
| | Technology | RRID: AB_2121214 |
| Rabbit polyclonal anti-Actin | Cell Signaling | Cat# 4970 |
| | Technology | RRID: AB_2223172 |
| Rabbit polyclonal anti-Ki67 | Abcam | Cat#: ab15580 |
| | | RRID: AB_443209 |
| Rabbit polyclonal anti-CTGF | Abcam | Cat#: ab6992 |
| | | RRID: AB_305688 |
| Rabbit polyclonal anti-WWTR1 | Sigma | Cat#hpa0077415 |
| Mouse monoclonal anti-Alpha-SMA | Invitrogen | Cat#14-9760-82 |
| | | RRID: AB_2572996 |
| Rabbit anti-IgG | Santa Cruz | Cat#: sc-2027 |
| | Biotechnology | RRID: AB_737197 |
| Mouse anti-IgG_{2B} | Santa Cruz | Cat#: sc-3879 |
| | Biotechnology | RRID: AB_737262 |
| Mouse monoclonal anti-p21 | Santa Cruz | Cat#: sc-6246 |
| | Biotechnology | RRID: AB_628073 |
| Rabbit polyclonal anti-IL1-β | Santa Cruz | Cat#: sc-7884 |
| | Biotechnology | RRID: AB_2124476 |
| Mouse monoclonal anti-hnRNPU | Santa Cruz | Cat#: sc-32315 |
| | Biotechnology | RRID: AB_627741 |
| Mouse monoclonal anti-Col1a1 | Santa Cruz | Cat#: sc-293182 |
| | Biotechnology | RRID: AB_2797597 |
| Rabbit polyclonal anti-pSer89-TAZ | Santa Cruz | Cat#: sc-17610-R |

| | Biotechnology | RRID: AB 671263 |
|---|---|---|
| Rabbit polyclonal anti-PDGFRb | Santa Cruz | Cat#: sc-432 |
| | Biotechnology | RRID: |
| | | AB_631068 |
| Rabbit polyclonal anti-TM7SF3 | Zick Lab | [14] |

Sequence-Based Reagents
Primers for qRT-PCR

| | | |
|---|---|---|
| Mouse TM7SF3 primers | Integrated | N/A |
| Forward: GTGTACCAGTACTTCCTGCCC | DNA | |
| (SEQ ID NO: 4) | Technologies | |
| Reverse: AGAGAAGGACACGGCTGTTTT | | |
| (SEQ ID NO: 5) | | |
| Mouse TGFB primers | Integrated | N/A |
| Forward: CTCCCGTGGCTTCTAGTGC | DNA | |
| (SEQ ID NO: 6) | Technologies | |
| Reverse: GCCTTAGTTTGGACAGGATCT | | |
| G (SEQ ID NO: 7) | | |
| Mouse COL1A1 primers | Integrated | N/A |
| Forward: | DNA | |
| GCTCCTCTTAGGGGCCA | Technologies | |
| CT (SEQ ID NO: 8) | | |
| Reverse: CCACGTCTCACC | | |
| ATTGGGG (SEQ ID NO: 9) | | |
| Mouse ACTA2 primers | Integrated | N/A |
| Forward: ATGCTCCCAGGGCTGTTTTCC | DNA | |
| CAT (SEQ ID NO: 10) | Technologies | |
| Reverse: GTGGTGCCAGATCTTTTCCAT | | |
| GTG (SEQ ID NO: 11) | | |
| Mouse Timp1 primers | Integrated | N/A |
| Forward: CTCAAAGACCTATAGTGCTGG | DNA | |
| C (SEQ ID NO: 12) | Technologies | |
| Reverse: CAAAGTGACGGCTCTGGTAG | | |
| (SEQ ID NO: 13) | | |
| Mouse MCP1 primers | Integrated | N/A |
| Forward: AGGTCCCTGTCATGCTTCTG | DNA | |
| (SEQ ID NO: 14) | Technologies | |
| Reverse: GCTGCTGGTGATCCTCTTGT | | |
| (SEQ ID NO: 15) | | |
| Mouse hnRNP U primers | Integrated | N/A |
| Forward: TCCCCTTAGAGGAC | DNA | |
| CGAGTT (SEQ ID NO: 16) | Technologies | |
| Reverse: GGGTTTTCAGCTGC | | |
| ATGTTT (SEQ ID NO: 17) | | |
| Mouse TEAD1 | Integrated | N/A |
| Forward: CCAGGATCCTCACAAG | DNA | |
| ACG (SEQ ID NO: 18) Reverse: | Technologies | |
| GAATGGGGGCTGTGACTG | | |
| (SEQ ID NO: 19) | | |
| Mouse TEAD2 | Integrated | N/A |
| Forward: CTGAGGACAGGGAA | DNA | |
| GACGAG (SEQ ID NO: 20) | Technologies | |
| Reverse: CTTCGAGCCAAAAC | | |
| CTGAAT (SEQ ID NO: 21) | | |
| Mouse TEAD3 | Integrated | N/A |
| Forward: GAGCTGATTGCCCGCT | DNA | |
| AC (SEQ ID NO: 22) | Technologies | |
| Reverse: TGTATGTGGCTGGACA | | |
| CCTG (SEQ ID NO: 23) | | |
| Mouse TEAD4 | Integrated | N/A |
| Forward: TCAAAACACCTACCO | DNA | |
| TGTCCA (SEQ ID NO: 24) | Technologies | |
| Reverse: GCCCTGCAGGAGAC | | |
| TCAA (SEQ ID NO: 25) | | |
| Mouse Ki67 primers | Integrated | N/A |
| Forward: ATCATTGACCGCTCCTTTAGG | DNA | |
| T(SEQ ID NO: 26) | Technologies | |
| Reverse: GCTCGCCTTGATGGTTCCT | | |
| (SEQ ID NO: 27) | | |
| Mouse Cyr61 primers | Integrated | N/A |
| Forward: GGAAAAGGCAGCTC | DNA | |
| ACTGAA (SEQ ID NO: 28) | Technologies | |
| Reverse: TCTGAACGATGCATT | | |
| TCTGG (SEQ ID NO: 29) | | |
| Mouse Vimentin primers | Integrated | N/A |
| Forward: TTTCTCTGCCTCTGC | DNA | |
| CAAC (SEQ ID NO: 30) | Technologies | |
| Reverse: TCTCATTGATCACCT | | |
| GTCCATC (SEQ ID NO: 31) | | |
| Mouse IL-6 primers | Integrated | N/A |
| Forward: | DNA | |
| CCAGAGATACAAAGAAATGATG | Technologies | |
| G (SEQ ID NO: 32) Reverse: | | |
| ACTCCAGAAGACCAGAGGAAAT | | |
| (SEQ ID NO: 33) | | |
| Mouse CLEC4 primers | Integrated | N/A |
| Forward: GGCCGGCCAGGGCTTTCTCCAG | DNA | |
| C ACAGTATGGACAC (SEQ ID NO: 34) | | |
| Reverse: | | |
| GCGGCCGCAGTCGAGATCGGAT | Technologies | |
| GAGACC GGCGTCTGGT (SEQ ID | | |
| NO: 35) | | |
| Mouse TNF-α primers | Integrated | N/A |
| Forward: | DNA | |
| CCAGACCOTCACACTCAGAT | Technologies | |
| C (SEQ ID NO: 36) Reverse: | | |
| CACTTGGTGGTTTGCTACGAC | | |
| (SEQ ID NO: 37) | | |
| Mouse 36B4 primers | Integrated | N/A |
| Forward: TGGCCAATAAGGTGCCAGCTGC | DNA | |
| G (SEQ ID NO: 38) | Technologies | |
| Reverse: CTTGTCTCCAGTCTTTATCAGCT | | |
| C (SEQ ID NO: 39) | | |
| Mouse TEAD1Ex5 primers | Integrated | N/A |
| Forward: AGCCAGATACATCAAACTCAGG | DNA | |
| A (SEQ ID NO: 40) | Technologies | |
| Reverse: CTTGGCAGTCTGATCCTTCA | | |
| (SEQ ID NO: 41) | | |
| Human TM7SF3 primers | Integrated | N/A |
| Forward: | DNA | |
| AGTGCTCTCAAGGTGGTTAC | Technologies | |
| (SEQ ID NO: 42) Reverse: | | |
| GAAACGGGTCCCAAACAAT | | |
| G (SEQ ID NO: 43) | | |
| Human TGFβ primers | Integrated | N/A |
| Forward: CTAATGGTGGAAACCCACAACG | DNA | |
| (SEQ ID NO: 44) | Technologies | |
| Reverse: TATCGCCAGGAATTGTTGCTG | | |
| (SEQ ID NO: 45) | | |
| Human ACTA2 primers | Integrated | N/A |
| Forward: CTATGAGGGCTATGCCTTGCC(S | DNA | |
| EQ ID NO: 46) | Technologies | |
| Reverse: GCTCAGCAGTAGTAACGAAGGA | | |
| (SEQ ID NO: 47) | | |
| Human Col1a1 primers | Integrated | N/A |
| Forward: GAGGGCCAAGACGAAGACATC | DNA | |
| (SEQ ID NO: 48) | Technologies | |
| Reverse: CAGATCACGTCATCGCACAAC | | |
| (SEQ ID NO: 49) | | |
| Human Timp1 primers | Integrated | N/A |
| Forward: AGAGTGTCTGCGGATACTTCC | DNA | |
| (SEQ ID NO: 50) | Technologies | |
| Reverse: CCAACAGTGTAGGTCTTGGTG | | |
| (SEQ ID NO: 51) | | |
| Human Desmin primers | Integrated | N/A |
| Forward: GAGACCATCGCGGCT | DNA | |
| AAGAAC (SEQ ID NO: 52) | Technologies | |
| Reverse: GTGTAGGACTGGATCT | | |
| GGTGT (SEQ ID NO: 53) | | |
| Human PNPLA3 primers | Integrated | N/A |
| Forward: CCTGTGGAATCTGCCATTGOGA | DNA | |
| (SEQ ID NO: 54) | Technologies | |
| Reverse: GAGCAGACACATCAGCACTOGA | | |
| (SEQ ID NO: 55) | | |
| Human PPARγ primers | Integrated | N/A |
| Forward: | DNA | |
| AGCCTGCGAAAGCCTTTTGGTG | Technologies | |
| (SEQ ID NO: 56) Reverse: | | |
| GGCTTCACATTCAGCAAACCTG | | |
| G (SEQ ID NO: 57) | | |
| Human Ki67 primers | Integrated | N/A |
| Forward: GAGAATCTGTGAATCT | DNA | |
| GGGTAA (SEQ ID NO: 58) | Technologies | |
| Reverse: CAGGCTTGCTGAGGG | | |
| AAT (SEQ ID NO: 59) | | |
| Human TEAD1Ex5 primers | Integrated | N/A |
| Forward: GGTAGGAATGAATTGATAGCCA | DNA | |
| GA (SEQ ID NO: 60) | | |
| Reverse: CTTTGCAGTCTGATCCTTTAG | Technologies | |
| (SEQ ID NO: 61) | | |
| Human CYR61 primers | Integrated | N/A |
| Forward: CAGGACTGTGAAGATGCGGT | DNA | |
| (SEQ ID NO: 62) | Technologies | |
| Reverse: GCCTGTAGAAGGGAAACGCT | | |
| (SEQ ID NO: 63) | | |
| Human CTGF primers | Integrated DNA | N/A |
| Forward: AGGAGTGGGTGTGTG | Technologies | |
| ACGA (SEQ ID NO: 64) | | |
| Reverse: CCAGGCAGTTGGCTC | | |
| TAATC (SEQ ID NO: 65) | | |
| Human 36B4 primers | Integrated DNA | N/A |
| Forward: | Technologies | |
| GGTGCCTCTGGAGATTTTAG | | |
| (SEQ ID NO: 66) Reverse: | | |
| CACTGGTCTCGGGCCCGAGA | | |
| A (SEQ ID NO: 67) | | |

Primers for PCR

| | | |
|---|---|---|
| Human TEAD1 AS primers | Integrated DNA | N/A |
| Forward: GGTGTCTAGTCACATT | Technologies | |
| CAGGT (SEQ ID NO: 68) | | |
| Reverse: TTATGAATGGCAGTGG | | |
| CCGA (SEQ ID NO: 69) | | |
| Human TEAD1 intron (RIP) primers | Integrated DNA | N/A |
| Forward: GCATCTGGATAGCATATCCCT | Technologies | |
| (SEQ ID NO: 70) | | |
| Reverse: TTCATGTGCCATAAAAAATGO | | |
| (SEQ ID NO: 71) | | |

SIRNA

| | | |
|---|---|---|
| SIRNA-mouse TM7SF3 | Horizon | Cat# M- |
| | Discovery | 057504-02- |
| | | 0005 |
| SIRNA-human TM7SF3 | Horizon | Cat# M- |
| | Discovery | 005745-00- |
| | | 0010 |
| SIRNA-human/mouse TEAD1DEx5 | Horizon | N/A (custom |
| Sense: AUUCCAAGCUAAAGGAUCAGAUU | Discovery | SIR |
| (SEQ ID NO: 72) | | NA |
| Antisense: UCUGAUCCUUUAGCUUGGAA | | |
| UUU (SEQ ID NO: 73) | | |
| SIRNA-Human TEAD1 | Horizon | M-012603- |
| | Discovery | 01- 0005 |
| Mouse siCON | Horizon | D- |
| | Discovery | 001210- |
| | | 05-05 |
| Human siCON | Horizon | Cat# D- |
| | Discovery | 001206- |
| | | 13-20 |
| SIRNA-Human hnRNPU | Horizon | Cat# M- |
| | Discovery | 013501-01- |
| | | 0005 |
| SIRNA-Human TAZ | Horizon | Cat# M- |
| | Discovery | 016083-00- |
| | | 0005 |
| SIRNA-Human CTGF | Horizon | Cat# M- |
| | Discovery | 012633-02- |
| | | 0005 |
| ASO 56 | Integrated DNA | N/A |
| /52MOErC/*/i2MOErA/*/i2MOErG/*/ | Technologies | |
| i2MOErC/*/12MOErT/*/i2MOErA/*/ | | |
| i2MOErA/*/i2MOErA/*/i2MOErA/*/ | | |
| i2MOErT/*/i2MOErA/*/i2MOErC/*/ | | |
| i2MOErA/*/i2MOErG/*/i2MOErA/*/ | | |
| i2MOErA/*/i2MOErT/*/i2MOErA/*/ | | |
| i2MOErC/*/i2MOErT/*/i2MOErC/*/ | | |
| 32MOErA/ | | |
| NC5 MOE SSO (22 mer) | Integrated DNA | N/A |
| /52MOErG/*/i2MOErC/*/i2MOErG/*/ | Technologies | |
| i2MOErA/*/12MOErC/*/i2MOErT/*/ | | |
| i2MOErA/*/i2MOErT/*/i2MOErA/*/ | | |
| i2MOErC/*/i2MOErG/*/i2MOErC/*/ | | |
| i2MOErG/*/i2MOErC/*/i2MOErA/*/ | | |
| i2MOErA/*/i2MOErT/*/i2MOErA/*/ | | |
| i2MOErT/*/i2MOErG/*/i2MOErG/*/ | | |
| 32MOErT/ | | |

### Cell Lines

Primary human HSCs (passages 3-5) were purified from livers using pronase perfusion and gradient centrifugation method. The HSCs and U2OS cells were cultured in DMEM supplemented with 10% FBS, 1% Pen/strep, and 1% Glutamax.

### Mice

Generation of TM7SF3 fl/fl mouse. A plasmid encoding the genomic tm7sf3 locus (PG00237_Z_6H11 [Tm7sf3] purchased from EuMMCR; Munich, Germany) was digested by AsiS restriction enzyme. This plasmid contains lox sites that flanking exons 5 and 6 of Tm7sf3. The linearized plasmid was then purified and transfected into C57B16 ES cells. Genomic DNA of transfected ES cells were screened by PCR for correct integration of the cassette with homology arms (19 kb). Positive ES cells were injected into a C57B16 blastocyst. The chimera mice were then cross bred with C57B16 mice. To remove LacZ and Neomycin resistance cassettes, the litters of the chimera mice were cross bred with Rosa-26-FLPe to generate TM7SF3^{fl/fl}. If mice. TM7SF3^{fl/fl} mice were cross-bred with Rosa-26-creert2 transgenic mice to generate: TM7SF3^{fl/fl} and Rosa-26-creert2::TM7SF3^{fl/fl} (TM7SF3 KO) mice. Animals were housed in an animal facility on a 12 hours/12 hours light/dark cycle at room temperature of 20-22 C with free access to food and water. Mice were in good health. All animal procedures were in accordance with UC San Diego and Institutional Animal Care and Used Committee-approved protocols and conformed to the Guide for Care and Use of Laboratory Animals of the National Institutes of Health. Eight-week-old male TM7SF3 KO and f/f mice were fed with a Tamoxifen diet (TD. 130859, ENVIGO) for 3 weeks. Male control and KO mice were fed with NCD or NASH diet (AIN76) for 4 weeks, and liver cells were isolated as described.

### Study Approval

All animal procedures were done in accordance with the University of California, San Diego Research Guidelines for the Care and Use of Laboratory Animals, and all animals were randomly assigned to cohorts when used.

### Method Details

### Hepatic Stellate Cell (HSC) and Non-Parenchymal Cell (NPC) Isolation

Mouse liver was sequentially perfused via the superior vena cava first with 40 mL of buffer SC-1 1 (NaCl 137 mM, KCl 5.37 mM, NaH₂PO₄·H₂O 0.64 mM, Na₂HPO₄ 0.85 mM, HEPES 10 mM, NaHCO₃4.2 mM, EGTA 0.5 mM, Glucose 5 mM, pH 7.4), then with buffer SC-2 (As SC-1, without EGTA and Glucose, but with CaCl₂·2H₂O 3.8 mM) with 0.5 mg/mL pronase 25 mL, and finally buffer SC-2 with 0.5 mg/mL collagenase D 25 mL. After digestion of the clipped liver tissue in a mixture containing DNase, collagenase D and pronase for 20 minutes, the cell suspension was filtered through a 70 mm cell strainer and centrifuged at 50×g for 1 minute at 4° C. The supernatant was collected and centrifuged at 900×g for 8 minutes and the pellet was washed once with GBSS buffer with CaCl₂·2H₂O 1.5 mM and HSCs were separated by Nycodenz 8.6% gradient (2000×g for 20 minutes). The obtained cells in the top layer are purified HSCs. The HSCs were washed once with GBSS buffer before cultured in DMEM media or used directly for RNA extraction.

The non-parenchymal cells that remain in the Nycodenz pellet were resuspended in SC-2 buffer and Pecoll 20% (600×g for 15 minutes) to remove parenchymal cells. Then the pellet was washed once with SC-2 buffer and resuspended with SC-2 and 28% OptiPrep, to enrich NPCs and remove HSCs. The tube was centrifuged at 1400×g for 25 minutes (4 C, low acceleration), and the cell layer between the 28% OptiPrep and cell suspension was collected. These cells were washed twice with centrifugation dilution solution (800×g for 10 minutes, 4 C), treated with red blood cell lysis buffer and seeded with culture medium (00% FBS, 1% Glutmax, and 1% penicillin/streptomycin) at 37° C. or used directly for RNA extraction.

### Isolation of Primary Hepatocytes

Primary hepatocytes were isolated as described previously [58] with some modifications. Briefly, mice were infused with a buffer SC-1 via the vena cava for 8 minutes (5 mL/minutes; Total 40 mL). After the color of the liver changed to a beige or light brown color, SC-2 buffer with 0.5 mg/ml collagenase D was perfused into the liver for 5 minutes (5 mL/minutes; Total 25 mL) After the appearance of cracking on the liver surface, perfusion was stopped, and the liver was excised into SC-2 buffer. Cells from digested livers were teased out, suspended in Buffer A, filtered through a 100 mm cell strainer, and centrifuged at 50×g for 1 minute at 4° C. The pellet was resuspended with Buffer SC-2 (no collagenase) and mixed with Percoll (adjusted to physiological ionic strength with 10×PBS) to a final concentration of 27% and centrifuged at 100×g for 10 minutes, 4° C. After removing the supernatant, the hepatocyte pellet was washed with Buffer SC-2 and resuspended in Williams Medium E containing 10% FBS and taken either for making liver organoids or were cultured on collagen-coated plates (GIBCO, Life Technologies) and antibiotics. After overnight incubation (16 hours), the culture medium was refreshed.

### Liver Organoid Culture

Primary hepatocytes, NPCs and HSCs from lean mouse were isolated as described above. About 1500-1600 hepatocytes, 500 stellate cells, and 750 Kupffer cells were seeded per well of a 96-well Ultra Low Attachment (ULA) plate from Corning (catalog #4515). The medium volume was 200 μl per well. The plating medium was WE+Supplement cocktail A+Dexamethasone+5% FBS. The plate was spinned at 50-100×g for 2 minutes and placed in the incubator. On day 3, 100 μl of the culture medium from each well were withdrawn gently by tilting the plate. Next 100 μl of fresh WE+supplement cocktail B+Dexamethasone, without serum. was added to each well. The medium was changed (only 50% with serum-free WE+supplement B) every 3rd day. Plate was spun at 50×g for 2 minutes each time medium replacement was carried out. Spheroids formation is visible after 3-5 days. At the end, spheroids from 8 wells were pooled for analysis.

siRNA Transfection

Recipient cells were treated with siRNA (20 pmol siRNA) using the RNAiMAX reagent. Control cells were treated with a non-targeting control siRNA. The siRNAs were mixed with RNAiMAX reagent in OptiMEM media, and then incubated for 15 minutes at room temperature. This mixture was then added to the cell media for 6 hours following by media replacement. The cells were transfected for 48 hours and then transfected again for additional 48 hours. The media was then replaced to serum free media overnight before treatment with TGFβ (5 ng/mL) as indicated.

### ASO Transfection or In Vivo Treatment

The ASO (500 nM) was mixed with lipofectamine RNAiMAX reagent and transfected into recipient cells with OptiMEM media. After 6 hours, the OptiMEM was replaced to DMEM media with 10% FBS, 1% Pen/Strep and 1% Glutamax. After 48 hours the cells were treated with TGFβ as described above. For in vivo delivery, the ASO 56 or ASO control (3 or 10 mg/Kg as indicated) were injected into the tail vein of recipient WT mice.

### Quantitative Reverse Transcription-Polymerase Chain Reaction (RT-PCR) and PCR Analysis

Total RNA was extracted from liver tissue, primary cells and cultured cells using TRIzol reagent according to the manufacturer standard protocol. First-strand cDNA was synthesized using Reverse transcriptase and random hexamers. Quantitative PCR was carried out in 10 ml reactions using SYBR Green mix on an StepOnePlus Real Time PCR system (ABI). Relative Gene expression was calculated as mRNA level normalized to that of a standard housekeeping gene (36B4) using the DDCT method. The specificity of the PCR amplification was verified by melting curve analysis of the final products using StepOne software (v2.3). Primer sequences were provided in key resources table.

### RNA-IP

For RNA-immunoprecipitation (RIP) experiments, human HSCs and U2-OS cells were homogenized in lysis TSE buffer (150 mM NaCl, 20 mM Tris-HCl, (pH 7.4), 2 mM EDTA, 1% Triton X-100, 0.1% SDS, 0.2 mM PMSF, with protease and phosphatase inhibitors) and 400 mg of cell lysate protein was incubated overnight with protein G beads and antibody complexes as indicated. The Beads were washed with TSE buffer three times, and RNA was eluted with TE buffer containing 10 mM DTT by shaking tubes in 37° C. for 30 minutes. RNA was extracted from the elution followed by reverse transcription reaction and a PCR with primers for TEAD1 pre-mRNA.

### Western Blot Analysis

Primary human HSCs, liver, were homogenized in RIPA buffer supplemented with protease and phosphatase inhibitors. Tissue lysates were subjected to Western blotting and proteins were detected by corresponding antibodies. The protein bands were analyzed using densitometry and Image J image analysis software, normalizing phosphorylated protein to total protein bands. Arbitrary densitometry units were quantified by ImageJ analysis.

### Histopathological Analysis, and Immunofluorescence Microscopy

Formalin-fixed, paraffin-embedded mouse liver sections were stained with Picrosirius (Sirius) red (Polysciences, #24901) to assess liver fibrosis. The stained sections were digitally scanned and 10 frames (×10 zoom) were taken from each section. The staining intensity was then analyzed by imageJ software.

For immunofluorescence staining, cells (5×104 cells/well; 24 wells/plate) were grown on glass coverslips; washed with PBS and fixed with 4% paraformaldehyde for 17 minutes at 22° C. Cells were permeabilized with 0.5% triton-X100 in PBS for 4 minutes, and thereafter blocked for 30 minutes in a blocking solution (Tris 10 mM, NaCl 150 mM, 0.5% Triton X-100, 10% normal Goat serum, 2% BSA, 1% Glycine, Ph 7.4). Cells were subjected to indirect immunofluorescence with the indicated antibodies diluted in blocking solution for 1 hour at 22° C. Cell were washed with PBS and primary antibodies were detected with Alexa488 Goat anti-rabbit secondary antibodies (Abcam, diluted 1:200 in PBS with 20% Normal horse serum) for 1 hour at 22° C. Cells were washed with PBS. Staining of nuclei was performed using and DAPI (0.5 μg/ml). The specimens were washed several times with PBS, and were mounted with Immu-mount (Thermo Scientific; Cheshire, UK) overnight on glass microscope slides. Images were obtained as single optical slides using Leica TCS SP8 confocal microscope SP8 LIGHTNING.

### Human HSC FACS Analysis

HSCs were dissociated from plate with trypsin, washed and blocked with FC Block and anti-CD16/32 solution. Viability and surface staining was done with a mix of PBS, FC-Block, and Zombie NIR. Fixation/Permeabilization working solution was added, and cells were stained with the intracellular antibody (Ki67) permeabilization buffer. After staining, cells were washed and were resuspended in 300 μL FACS Buffer.

### HSC Proliferation Assay

Proliferation of human HSCs was measured using the WST-1 cell proliferation kit as previously described [23]. Briefly, HSCs in 96-well plates were transfected with siRNAs as described above. After 4 days, the cells were cultured overnight in fresh DMEM without FBS followed by culturing in DMEM and TGFβ (5 ng/mL) for an additional 24 hours. The media were changed to fresh DMEM, and the WST-1 reagent was added for 4 hours, followed by assaying absorbance at 440 nm with a plate reader.

### Measurement of Hydroxyproline Content of Liver Tissue

Hydroxyproline liver content was measured as previously described [59]. Briefly, liver tissues were incubated with 6N hydrochloric acid for 30 minutes in 110° C. followed by homogenization. Then, samples were hydrolyzed by incubation with 6N hydrochloric acid at 110° C. for 16 hours followed by neutralization with sodium hydroxide. Liver hydrolysates were oxidized using chloramine-T, followed by incubation with Ehrlich's perchloric acid reagent for color development. Absorbance was measured at 560 nm, and hydroxyproline quantities were calculated by reference to standards processed in parallel. Results are expressed as ng per mg liver weight.

### Heparin Beads for Secreted CTGF Protein

Secreted CGTF protein was pull down with heparin beads as previously described [60]. Conditioned media from human HSCs was incubated with heparin-agarose beads (Cat #H6508 from Sigma) at 4° C. overnight with rotation. Beads were washed 3 times in PBS, boiled in Laemmli sample buffer and subjected to Western blotting as described above. CTGF blot densitometry was normalized to total cellular protein.

### Quantification and Statistical Analyses

No blinding experiments were performed. No samples or data were excluded from the study for statistical purposes. Each in vitro experiment was independently performed in duplicate or triplicate to ensure reproducibility. Group sizes of 5 mice or greater were sufficient to reach a statistical power of at least 80%. Mice were assigned at random to treatment groups for all mouse studies. Tests used for statistical analyses are described in the figure legends. To assess whether the means of two groups are statistically different from each other, unpaired two-tailed Student's t test was used for statistical analyses, all data passed the normality test using Prism9 software (GraphPad software v9.0; Prism, La Jolla, CA). p values of 0.05 or less were considered to be statistically significant. Degrees of significance are indicated in the figure legends. For the results of glucose and insulin tolerance tests, statistical comparisons between the two groups at each time point were performed with unpaired two-tailed Student's t test. Data are presented as the means±s.e.m. The significance of differences between groups was evaluated using analysis of variance. In figures, asterisks denote statistical significance (*, P<0.05; **, P<0.01; ***, P<0.001; ****, P<0.0001). Statistical analysis was performed in Graph Pad PRISM 9.

### Results

### TM7SF3 Regulates Liver Fibrosis and Activation of HSCs

TM7SF3 is a nuclear seven-transmembrane protein that modulates alternative splicing [14]. Since TM7SF3 is well expressed in the liver [15], the role of TM7SF3 in HSC activation and liver fibrogenesis was assessed.

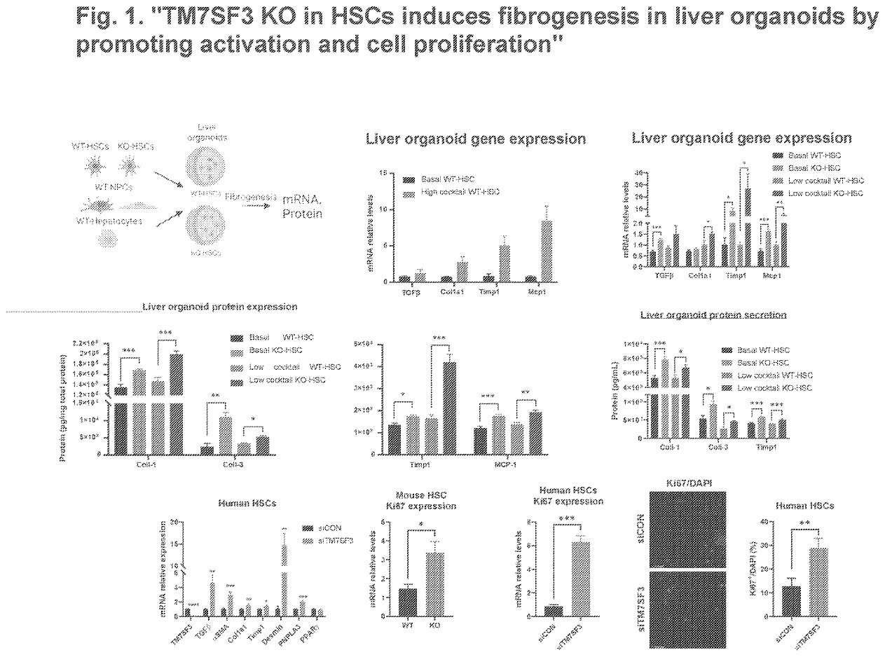

To initially evaluate this, a liver organoid system made up of WT-hepatocytes and Non-parenchymal cells (NPCs), with HSCs from WT (WT-HSCs) mice or whole-body TM7SF3KOs (KO-HSCs; FIG. 1A) was generated. Over a 14-day period in culture, these cells organize into spheroid-shaped structures on 24-well ultra-low adhesion plates. As opposed to primary cultures of hepatocytes or HSCs alone, these liver organoids contain multiple liver cell types, express much higher levels of fibrogenic and inflammatory genes [16]. To simulate the NASH process, these liver organoids were treated with a cocktail of fatty acids (FAs; Palmitate 0.25 mM, Oleate 0.25 mM), fructose (10 mM), and lipopolysaccharide (LPS; 1 μg/ml)[17]. As previously reported, this cocktail robustly activates fibrogenic and inflammatory gene expression in these liver organoids. Next it was assessed whether the use of TM7SF3 KO HSCs would further exaggerate fibrogenic and inflammatory programs. Since the cocktail cause such strong responses (FIG. 1B), which might mask any increase due to the KO HSCs, the liver organoids were treated with a low dose of the cocktail (FAs; Palmitate 0.1 mM; Oleate 0.1 mM, fructose 2 mM, and LPS; 100 ng/ml) plus the KO HSCs. As seen in FIG. 1C, in the basal condition incorporation of KO-HSCs led to greater expression of TGFβ, Timp1, and MCP-1 compared to organoids with WT-HSCs. The low-dose NASH-inducing cocktail had only small effects to stimulate fibrogenic and inflammatory gene expression, while inclusion of the KO HSCs resulted in greater gene and protein expression of an array of fibrogenic genes compared to WT organoids (FIGS. 1C&D). Consistent with this, the organoids containing the KO HSCs demonstrated a further increase in TIMP1, and the inflammatory genes TGFβ, and MCP-1 (FIGS. 1 C&D). This experimental design also led to an increase in ECM protein production and secretion in the KO HSCs-containing organoids (FIG. 1E).

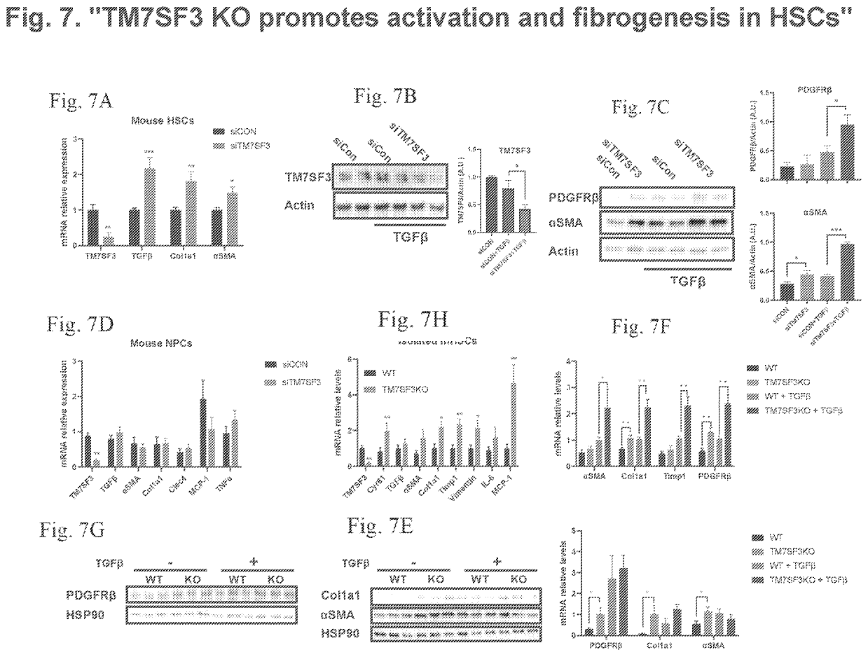

The liver organoid system contains a variety of cell types, and to specifically examine the role of TM7SF3 in the activation of HSCs, the TM7SF3 gene in primary human and mouse HSCs was silenced using siRNAs. The knockdown of TM7SF3 was confirmed using qPCR, with an about 90% reduction in TM7SF3 expression compared with scrambled-siRNA transfected control HSCs (FIG. 1F). It was found that TM7SF3 KD mouse and human HSCs displayed significantly greater fibrogenic gene and protein expression compared with control HSCs (FIGS. 1F and 7A-C). Importantly, TM7SF3-KD in NPCs that do not contain HSCs does not affect fibrogenic or inflammation-related gene expression. To further establish the role of TM7SF3 in HSC activation, HSCs from TM7SF3KO mice were studied after feeding a NASH diet for only one month. As shown in FIG. 7E, isolated HSCs from TM7SF3KO mice exhibited elevated expression of various fibrogenic and inflammatory genes. In addition, when the TM7SF3KO HSCs were treated with TGFβ, greater gene and protein expression levels were observed compared to WT (FIGS. 7F-H).

A feature of HSC activation is increased proliferation [18]. Indeed, it was shown that TM7SF3 KO in mouse and TM7SF3 KD in human HSCs increases Ki67 mRNA levels compared to WT controls (FIGS. 1G&H) along with an increase in Ki67 positive stained cells in human TM7SF3 KD HSCs (FIG. 1I). Taken together, these results indicate that TM7SF3 inhibits HSC differentiation and activation both in human and mouse cells and reduces fibrogenesis and inflammation in liver organoids.

### TM7SF3 Regulates HSC Activation by Modulation of TEAD1 Alternative Splicing

Alternative splicing can regulate liver physiology by splicing a variety of different genes, such as the transcription factor TEAD1 [19]. Additionally, since analyses of chromatin-defined enhancer sequences during HSC activation identified enrichment of TEAD motifs [20], we tested whether TEAD1 mediates the effects of TM7SF3 KD on HSCs.

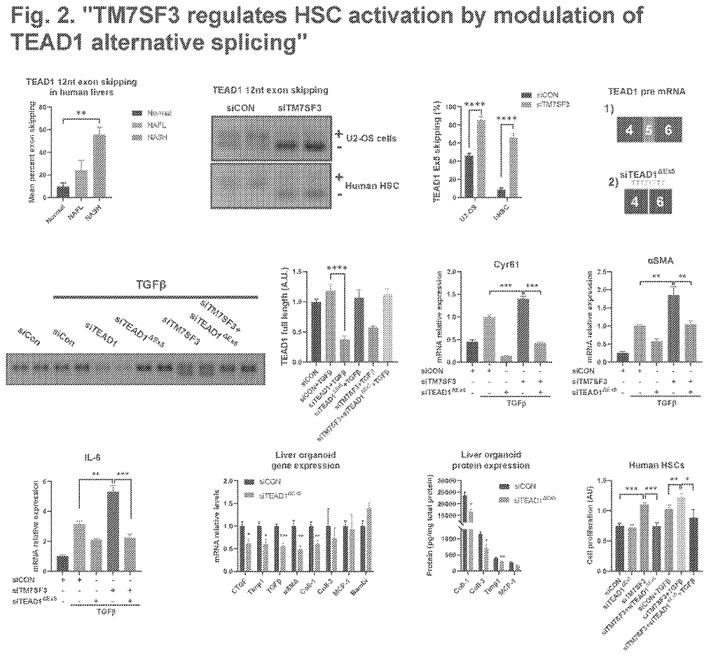

TEAD1 pre-mRNA contains a 12 nt microexon (exon 5; Human GRCh38/hg38: chr11:12,878,889-12,878,900) that encodes four amino acids just downstream of the DNA binding helix H3 [19]. Interestingly, the expression of TEAD1 in which this Exon is spliced-out (termed TEAD1^{ΔEx5}) is elevated in human livers from NASH patients compared to livers from healthy and NAFL patients (FIG. 2A). Exon 5 in TEAD1 includes a conserved serine residue, which, when phosphorylated, strongly inhibits TEAD1's DNA-binding ability [19]. Thus, excluding this microexon should increase TEAD1 transcriptional activity [19]. Indeed, it was found that TM7SF3 KD in human HSCs and U2-OS cells promotes Exon 5 skipping, leading to an increase in TEAD1^{ΔEx5} (FIG. 2B&C). Next, it was examined whether TEAD1^{ΔEx5} mediates the effects of TM7SF3 KD on HSC activation. An siRNA was designed, ‘siTEAD1^{ΔEx5}’, that targeted the exon-exon junction where the inhibitory Exon 5 is spliced-out (FIG. 2D). To test the efficiency and specificity of this customized siRNA, human HSCs were transfected with either siRNA against TEAD1, TEAD1^{ΔEx5}, or siCON. As shown in FIG. 2E, siTEAD1^{ΔEx5} did not inhibit TEAD1 expression, where siTEAD1 caused an about 60% decrease (FIG. 2F). However, siTEAD1^{ΔEx5} efficiently silenced the alternatively spliced active form of TEAD1 that was induced by TM7SF3 KD (FIG. 2E). Additionally, the ability of siTEAD1^{ΔEx5} to specifically inhibit the active form of TEAD1^{ΔEx5} was shown by a decrease in the TEAD1 target gene Cyr61 [21](FIG. 2G). While TM7SF3 KD caused an increase in the TEAD1 target gene, Cyr61, co-silencing both TM7SF3 with siTEAD1^{ΔEx5} completely abolished this effect (FIG. 2G), siTEAD1^{ΔEx5} also repressed the TM7SF3 KD-induced expression of the HSC activation marker αSMA and the fibrogenic cytokine IL-6 (FIG. 2H&I).

Then the ability of siTEAD1^{ΔEx5} to reduce fibrosis was examined in the more complex liver organoid system. Liver organoids were transfected every three days with siTEAD1^{ΔEx5} or siCON, during the 14-day culture with the NASH-inducing cocktail. As shown in FIG. 2J, inhibition of TEAD1^{ΔEx5} results in a significant decrease in fibrogenic gene and protein expression but didn't change the expression of the inflammation marker MCP-1 (FIG. 2J&K). Interestingly, expression of Bambi, a marker for the HSC quiescent state [22], was greater when TEAD1^{ΔEx5} was inhibited by siTEAD1^{ΔEx5} compared to control (FIG. 2J). Finally, the effects of TM7SF3 and TEAD1^{ΔEx5} on HSC proliferation were evaluated. TM7SF3 KD promotes HSC proliferation, as measured by a previously established assay [23](FIG. 2L). Inhibition of TEAD1^{ΔEx5} in the basal state did not affect HSC proliferation. However, co-silencing both TM7SF3 and TEAD1^{ΔEx5}, in basal or TGFβ-treated human HSCs, eliminates the effect of TM7SF3 KD to enhance cell proliferation (FIG. 2L). Together, these data suggest that active TEAD1 promotes the development of NASH through HSC activation and mediates the effects of TM7SF3 KD to stimulate HSC activation and proliferation.

TM7SF3 Inhibits TEAD1 Alternative Splicing Via the hnRNPU Splicing Factor

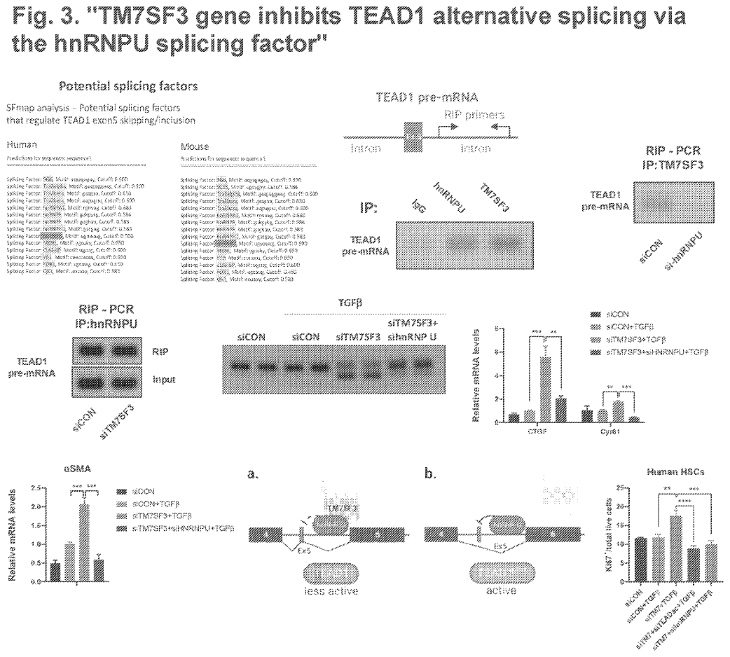

Regulation of alternative splicing is generally achieved by the interaction of splicing factors with pre-mRNA sequences [24]. An RNA recognition motif (RRM) was not identified in TM7SF3 protein, but a previous study showed that TM7SF3 can bind to RNA splicing factors or other RNA-binding proteins [14]. To identify the splicing factor which directly regulates TM7SF3 KD-induced TEAD1 alternative splicing, the list of TM7SF3 binding proteins [14] was examined for potential splicing factors that have an RNA binding motif sequence 100 bp up- or downstream of the TEAD1 Exon 5. As shown in FIG. 3A, the heterogeneous nuclear ribonucleoprotein U (hnRNPU) is the only splicing factor that has a binding motif sequence close to the TEAD1 microexon and also associates with TM7SF3 protein [14].

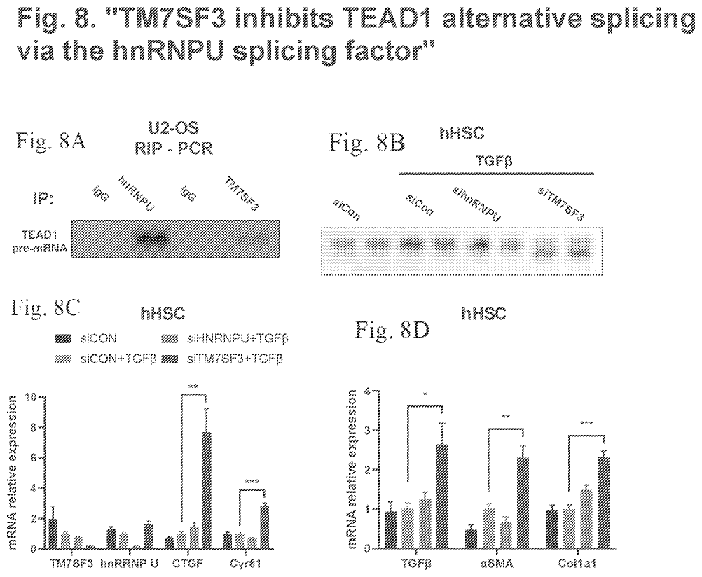

To further verify the interaction of hnRNPU with TEAD1 pre-mRNA, RNA immunoprecipitation (RIP) was performed with antibodies against hnRNPU and TM7SF3 in human HSC extracts. For TEAD1 pre-mRNA detection, primers that are downstream to the TEAD1 Exon 5 were designed (FIG. 3B). As shown in FIG. 3C, it was observed significant enrichment of TEAD1 pre-mRNA with hnRNPU and TM7SF3 antibody precipitation compared to IgG control. TEAD1 pre-mRNA enrichment was also detected with the same RIP conditions in U2-OS cells (FIG. 8A). Moreover, silencing hnRNPU in human HSCs reduced the amount of TEAD1 pre-mRNA pulled down with antibodies against TM7SF3 compared to control (FIG. 3D). In contrast, when TM7SF3 was silenced in human HSCs the binding of hnRNPU to TEAD1 pre-mRNA was not affected compared to controls (FIG. 3E). It was hypothesized that binding of hnRNPU to TEAD1 pre-mRNA leads to skipping of Exon 5. To further test this hypothesis, siRNA was used to KD hnRNPU and TM7SF3 separately or both together in human HSCs. TM7SF3 KD promotes exon 5 exclusion while no alternate splicing of exon 5 is seen with hnRNPU KD (FIG. 8B). Similarly, TM7SF3 KD promotes TGFβ-induced TEAD1 activity and HSC activation while hnRNPU KD has no effect (FIGS. 8C&D). However, hnRNPU KD reduced the ability of TM7SF3 KD to induce the splicing of TEAD1 into its active form (FIG. 3F). Furthermore, co-silencing of TM7SF3 and hnRNPU reduces the expression of direct TEAD1 target genes (FIG. 3G) as well as HSC activation (FIG. 3H) induced by TM7SF3 KD alone. These results suggest that TM7SF3 associates with hnRNPU and inhibits its activity to promote TEAD1 Exon 5 skipping (FIG. 3Ia). When TM7SF3 is depleted, hnRNPU is no longer inhibited and as a result, TEAD1^{ΔEx5} is increased and promotes HSC activation (FIG. 31b). Finally, si-hnRNPU in human HSCs inhibited cell proliferation promoted by TM7SF3 KD to the same degree as siTEAD1^{ΔEx5}, as measured by Ki67 positive cells (FIG. 3K). All of these results indicate that hnRNPU binds to TEAD1 pre-mRNA and splices out the inhibitory microexon (FIG. 31b).

### Antisense Oligomers Targeting TEAD1 Pre-mRNA Deactivate HSCs

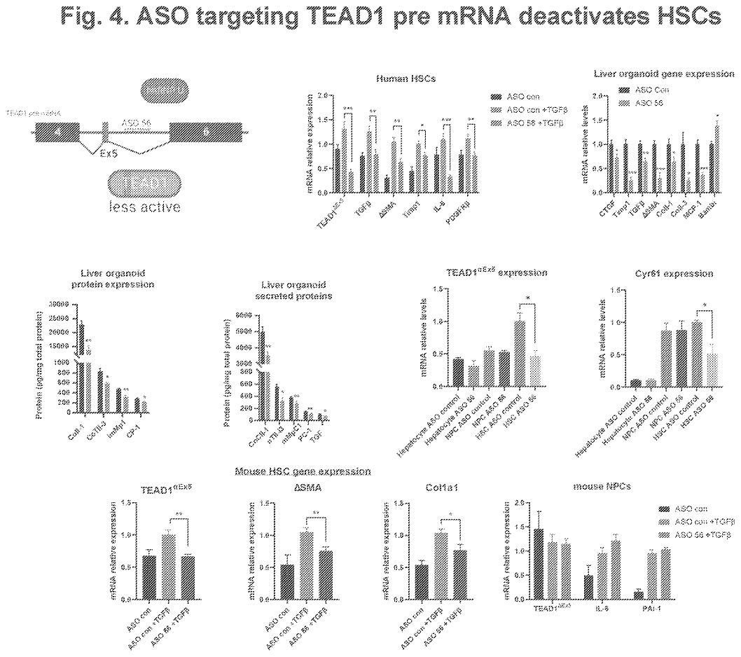

To further assess the impact of TEAD1 alternative splicing on HSC activation and NASH progression, an antisense oligomer (ASO) was designed that targets the hnRNPU binding motif on intron 5 just downstream of exon 5 (ASO 56) (FIG. 4A). Primary human HSCs were treated with ASO 56 or control ASO for 48 h followed by treatment with TGFβ for 24 hours. As shown in FIG. 4B, treatment of control HSCs with TGFβ increased the expression of TEAD1^{ΔEx5}, TGFΔ, αSMA, Timp1, PDGFRβ, and IL-6. However, pretreatment of HSCs with ASO 56 reduced TEAD1^{ΔEx5}, TGFβ, αSMA, Timp1, IL-6, and PDGFRβ (FIG. 4B). ASO 56 strongly inhibits the expression of TEAD1^{ΔEx5} after TGFβ stimulation indicating the efficacy of this ASO approach at targeting hnRNPU and also showing that the effects of TGFβ to increase TEAD1^{ΔEx5} expression are mediated through hnRNPU.

The motif binding of hnRNPU to mouse and human TEAD1 pre-mRNA is identical, and, therefore, we also tested the effect of ASO 56 in a mouse system. First, it was assessed the effect of ASO 56 on reducing the fibrogenesis program in the liver organoid system. ASO 56 or control ASO were delivered to liver organoids by transfection every three days during induction with the NASH cocktail as described above. ASO 56 treatment inhibited fibrogenic gene (FIG. 4C) and protein expression (FIGS. 4D&E). In contrast, the expression of the quiescent HSC marker, Bambi, was greater with ASO 56 treatment compared to control (FIG. 4C). Finally, gene and protein expression of MCP-1 was reduced in liver organoids treated with ASO 56 compared to control (FIGS. 4C-E).

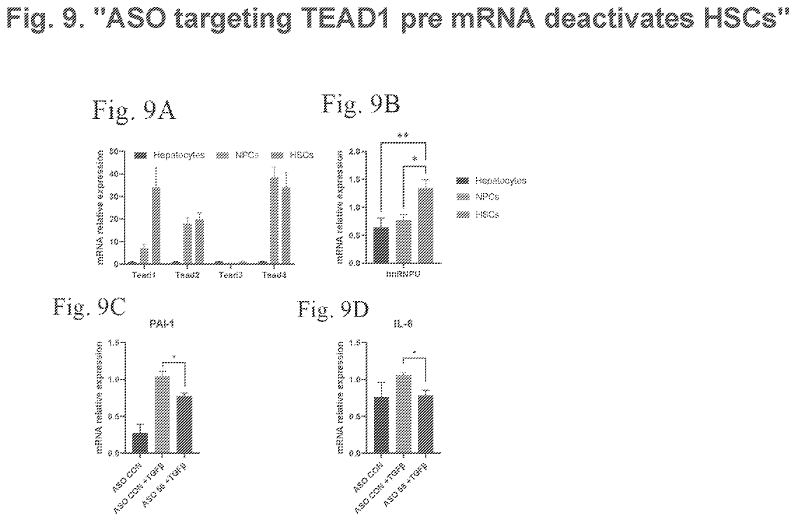

ASOs injected into mice are concentrated in the liver [25], and therefore, the ability of ASO 56 to inhibit TEAD1 alternative splicing in vivo was evaluated. A a single ASO dose (10 mg/Kg; IV) was injected and, 7 days later, the mice sacrificed for mRNA analysis of hepatocytes, NPCs (depleted of HSCs), and HSCs. While ASO 56 did not affect TEAD1 alternative splicing in hepatocytes or NPCs, ASO 56 reduced TEAD1^{ΔEx5} by about 50% in HSCs (FIG. 4F). Additionally, ASO 56 reduced the basal expression of the TEAD1 target gene, Cyr61 in HSCs compared to control ASO (FIG. 4G). Interestingly, in the liver, among the TEAD family transcription factors (1-4), it was found that only TEAD1 expression is enriched in HSCs compared to hepatocytes and NPCs (FIG. 9A). Additionally, the expression of hnRNPU is higher in mouse HSCs compared to hepatocytes and NPCs (FIG. 9B). The finding that ASO 56 affected HSCs but not NPCs was also validated in vitro. Primary mouse HSCs and NPCs (depleted of HSCs) were treated with ASO 56 or control ASO for 48 hours followed by treatment with TGFβ for 24 hours. As shown in FIG. 4H, ASO 56 reduced exon 5 skipping back down to the basal or control state. Moreover, treatment with ASO 56 inhibited TGFβ-induced expression of αSMA and Col1a1 (FIG. 4H). A similar reduction was seen in plasminogen activator inhibitor 1, PAI-1, and IL-6, which are upregulated during HSC activation [26-28]. In contrast, ASO 56 treatment did not affect TEAD1 alternative splicing or TGFβ-induced PAI-1 or IL-6 expression in the NPCs (FIG. 4I). Thus, hnRNPU-mediated alternative splicing of TEAD1 occurs in HSCs, but not in hepatocytes (FIG. 4F) or other NPC types, explaining why inhibiting TEAD1^{ΔEx5} expression reduces the activation of HSCs both in vitro and in vivo. The selectivity of hnRNPU splicing of TEAD1 exon 5 supports the potential therapeutic utility of a long acting version of ASO 56 in humans.

ASO Targeting TEAD1 Pre mRNA Deactivates HSCs In Vivo with Anti-NASH Effects

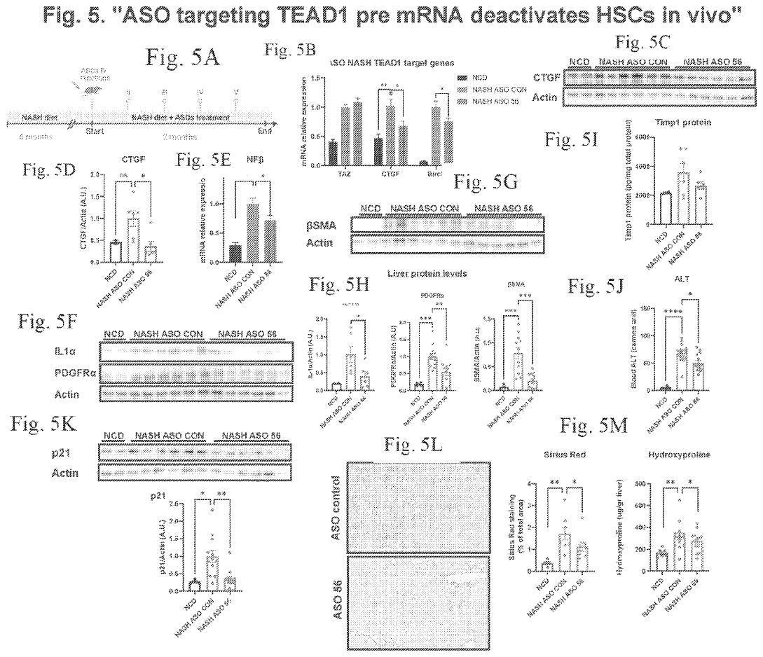

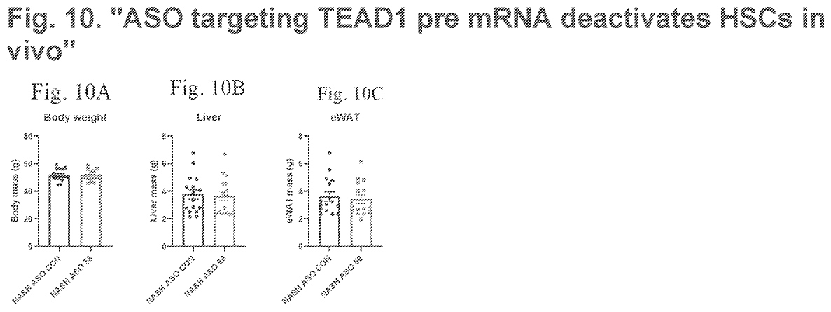

To assess the ability of ASO 56 to ameliorate the NASH phenotype in vivo, mice were placed on a NASH diet along with ASO 56 treatment according to the protocol in FIG. 5A. At eight weeks of age, male mice were fed a Western Diet (WD; AIN-76A) for six months. During the last two months of WD, the mice were treated with five IV injections of ASO 56 or control ASO (3 mg/Kg) (FIG. 5A). As shown in FIG. 10A, ASO 56 treatment did not affect body weight, liver mass (FIG. 10B), or fat mass (FIG. 10C).

ASO 56 treatment led to reduced expression of the TEAD1 target genes, CTGF and Birc5, while the NASH-associated increase in TAZ expression was unaffected (FIG. 5B-D). Moreover, liver TNF$ expression (FIG. 5E) and IL-1β protein levels were reduced by treatment with ASO 56 (FIG. 5F&H). Additionally, mice fed the WD and treated with control ASO showed a robust increase in expression of the HSC activation marker, PDGFRβ, and αSMA compared to NCD (FIG. 5F-H). At the same time, ASO 56 treatment led to a near complete reversal of this induction (FIG. 5F-H). Consistent with this, ASO 56 treatment also normalized Timp1 expression (FIG. 5I), improved blood ALT levels (FIG. 5J), and reduced the senescence marker, p21 [29], (FIG. K) when compared to NASH ASO control mice.

Liver fibrosis was also substantially improved. Thus, after 6 months of WD, the ASO control-treated mice developed the expected degree of fibrosis as shown by increased Sirius red staining and biochemically measured hydroxyproline concentration (FIGS. 5 L&M). In the ASO 56 treated mice, collagen content, as measured by Sirius red staining and liver hydroxyproline content, was markedly reduced (FIGS. 5 L&M). Together, these results indicate that in vivo ASO 56 treatment leads to decreased fibrogenesis in HSCs with reduced collagen deposition in the liver.

### TM7SF3 KD Regulation of HSC Activation is Mediated by TAZ

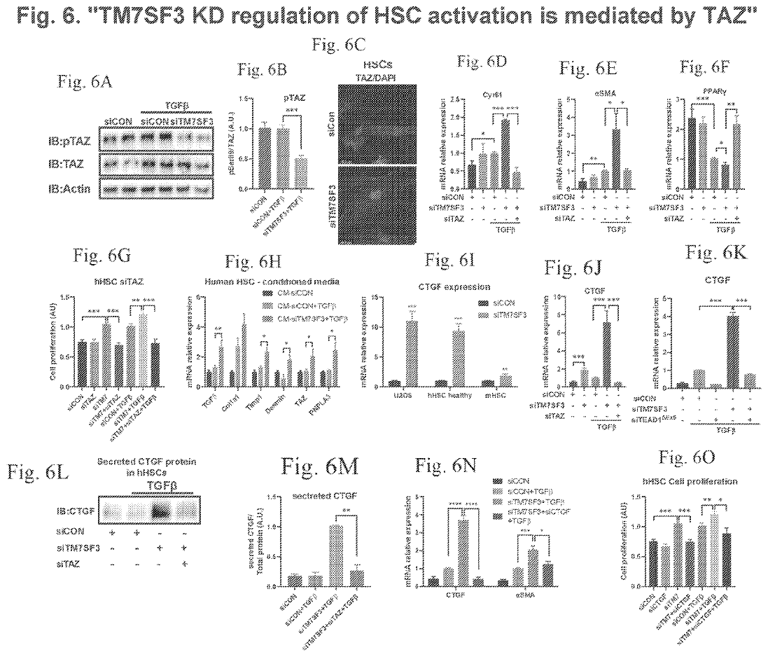

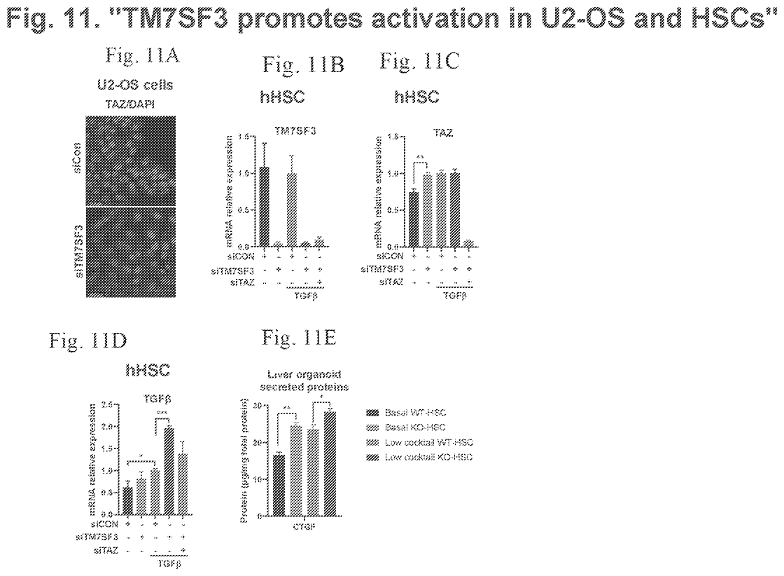

Next, the effect of TM7SF3 KD on the TEAD1 co-activator, TAZ [30], was studied. When the Hippo signaling pathway is inactive, TAZ is not phosphorylated on its inhibitory Serine 89 (Ser89) site [31]. Unphosphorylated TAZ translocates to the nucleus and associates with TEAD1 to promote gene transcription [31]. It was found that TM7SF3 KD reduces TAZ ser89 phosphorylation (FIGS. 6A&B), with a corresponding increase in nuclear localization of TAZ in human HSCs (FIG. 6C) and U2-OS cells (FIG. 11A). Next, it was tested whether TAZ mediates the effect of TM7SF3 KD on HSC activation. To address this question, we silenced TM7SF3 alone or with TAZ KD (FIGS. 11B&C) in human HSCs. As seen in FIG. 6D&11D, TGFβ induces the expression of the TAZ/TEAD1 target gene, Cyr61 [32], with a further increase in TM7SF3 KD HSCs. TAZ KD completely abolished the effect of TM7SF3 KD to induce Cyr61, αSMA (FIG. 6D&E), and TGFβ expression (FIG. 11E). Additionally, while TM7SF3 KD led to reduced expression of PPARγ compared to control, TAZ KD in TM7SF3 KD HSCs, fully prevented the TM7SF3 KD-mediated reduction in PPARγ levels (FIG. 6F). Finally, TAZ KD prevented the increase in HSC proliferation caused by TM7SF3 KD (FIG. 6G).

It was examined whether the TM7SF3 KD-induced activation of HSCs induces the secretion of fibrogenic factors. TM7SF3 KD human HSCs were treated with TGFβ for 24 hours. Conditioned media (CM) was then collected and incubated with naïve HSCs for 24 hours. As shown in FIG. 6H, CM from TM7SF3 KD-HSCs induced fibrogenic gene expression in naïve HSCs compared to controls.

Connective tissue growth factor (CTGF) is a secreted matricellular protein that synergizes with the action of TGFβ [33]. Silencing of TM7SF3 in U2-OS, human, and mouse HSCs induces CTGF expression (FIG. 6I) and that concomitant TM7SF3 plus TAZ or TEAD1^{ΔEx5} KD completely blocks this effect (FIG. 6J&K). Additionally, TM7SF3 KD promotes the secretion of CTGF in human HSCs (FIG. 6L&M) and liver organoids generated with TM7SF3KO HSCs (FIG. 11G) and this effect was blocked by TAZ KD (FIGS. 6L&M). Lastly, TM7SF3 KD-induced αSMA expression (FIG. 6N) and HSC proliferation (FIG. 6O) and these effects were blocked by co-treatment with siRNA against CTGF compared to control. These data indicate that TM7SF3 regulates the expression and nuclear localization of TAZ. When TM7SF3 is deleted, non-phosphorylated (Ser89) nuclear-localized TAZ promotes expression and secretion of fibrogenic genes, including CTGF, to mediate HSC fibrogenesis.

### Discussion

HSC activation is a critical step in the pathophysiology of NASH [34], yet the underlying molecular elements that trigger and drive HSC activation remain incompletely understood. TM7SF3 is a 7 transmembrane nuclear localized protein that participates in alternate mRNA splicing. Here it was shown that the TM7SF3 pathway regulates mRNA alternative splicing in HSCs and that silencing TM7SF3 accelerates proliferation and activates the fibrogenic program in human and mouse HSCs, as well as in liver organoids. Moreover, the Hippo pathway was identified as the mediator of the TM7SF3 effect on HSC activation and liver fibrosis. Finally, an ASO, which inhibits the Hippo pathway specifically in HSCs, deactivates HSCs and reduce liver fibrosis induced by a NASH diet in vivo. Thus, these data reveal a new role for TM7SF3 in controlling the Hippo pathway, HSC activation, and the development of fibrosis due to NASH.

In this study, it was found that TM7SF3 controls the Hippo pathway in HSCs by at least two mechanisms. Thus, TM7SF3 KD results in an increase in alternative splicing which removes exon 5 of TEAD1. Exon 5 contains an inhibitory serine phosphorylation site, so that full-length TEAD1 is relatively inactive, whereas, TEAD1 missing exon 5 (TEAD1^{ΔEx5}) is fully active. TEAD1 functions as a transcription factor within the Hippo pathway and is highly conserved and characterized by the TEA DNA binding domain [30]. TEAD1 induces transcription by interacting with its coactivators TAZ and/or YAP [30]. Among the target genes are well known fibrogenic factors, such as CTGF, CYR61, etc. [35]. Our data demonstrate that TEAD1^{ΔEx}5 deletion, or inhibition of TEAD1 activity, inhibits the expression of a broad range of fibrogenic genes in HSCs, liver organoids, and reduces fibrosis in NASH mice. The present approach was to either silence the active form of TEAD1 (TEAD1^{ΔEx5}) by siRNA or to inhibit its expression in HSCs using specifically targeted ASOs. These approaches reverse HSC activation induced by siTM7SF3, or TGFβ in vitro, or by a NASH diet in vivo. Of note, in all these conditions, the expression of full length, inactive, TEAD1 did not change, indicating that only the alternatively spliced TEAD1^{ΔEx5} participates in HSC stimulation. It is of interest that TGFβ stimulation causes an increase in the active TEAD^{ΔEx5} form and this effect is inhibited by ASO 56-mediated interference with hnRNPU activity. Although the mechanism underlying TGFβ stimulation of TEAD^{ΔEx5} is still unclear, the results with ASO 56 treatment demonstrate that the TGFβ effect is dependent on hnRNPU.

HNRNPU is a nuclear matrix protein involved in RNA processing and transport, as well as regulation of three-dimensional chromatin structure and gene transcription [36]. At the molecular level, hnRNPU is the splicing factor that interacts with TM7SF3 and binds to the intronic sites that flank exon 5 of TEAD1 pre-mRNA which contain the inhibitory serine phosphorylation site. When TM7SF3 is deleted, hnRNPU promotes alternative splicing of TEAD1 by excluding Exon 5. In the presence of TM7SF3, the activity of hnRNPU is inhibited, favoring production of inactive full length TEAD1. The RNP-IP data showed that both TM7SF3 and hnRNPU precipitate TEAD1 pre-mRNA in HSCs. Interestingly, the interaction of TM7SF3 with TEAD1 pre-mRNA is hnRNPU-dependent, but silencing TM7SF3 did not affect hnRNPU binding to TEAD1 pre-mRNA. Therefore, it was concluded that TM7SF3 inhibits the activity of hnRNPU. A recent study showed that Hepatocyte-specific hnRNPU knockout accelerates the development of NASH in vivo [37]. The present results suggest a role of hnRNPU exclusively in HSCs as a splicing factor promoting the Hippo pathway and fibrogenic gene expression.

The TEA domain comprises a three-helix bundle, where the H3 helix provides the interface for DNA binding [19], and exon 5 contains four amino acids just downstream of the DNA binding helix H3. Amongst these four amino acids is a conserved serine residue, which, when phosphorylated by Protein kinase A or C, strongly inhibits TEAD1's DNA-binding ability [38, 39]. Thus, the inclusion of exon 5 should serve as an alternate mechanism for reducing TEAD1 activity independent of its interaction with co-activators. To blunt TEAD1 activity, an ASO was designed which specifically inhibits the binding of hnRNPU to the TEAD1 pre-mRNA just upstream of exon 5. In in vitro studies using HSCs, as well as in in vivo studies in which the ASOs were used in vivo to treat mice on a NASH inducing diet, it was found that the ASO robustly reduced the expression of TEAD1^{ΔEx5} and its downstream fibrogenic target genes. In vivo, this resulted in amelioration of the liver fibrosis typically seen in NASH.

The present studies show that the TM7SF3/TEAD1/hnRNPU mechanism is operate in human HSCs, just as in mouse HSCs. To further demonstrate the relevancy of TEAD1^{ΔEx5} in the development of NASH disease, it was found that in human livers the expression of TEAD1 and the specific alternative splicing event of exon 5 exclusion is higher in livers of NASH patients when compared to normal or NAFL livers. Indeed, several reports support the involvement of TEAD1 in liver fibrosis. For example, Liu showed that motifs for the TEAD transcription factor were enriched in activated HSC enhancers with greater acetylation of histone 3 Lysine 27, which indicates higher transcription activity [20].

ASOs have been used as therapeutic agents for two decades; e.g., to provide a splicing correction in Spinal Muscular Atrophy (SMA), an important inherited cause of infant mortality [40]. Early work on the distribution of ASOs in animals showed that the liver and kidney accumulate higher concentrations than other organs [41]. Moreover, phosphorothioate oligonucleotide (PS) ASOs, as used in the present study, accumulate in liver NPCs [42]. Of note, the ASO doses used in the present in vivo studies were 3 mg/kg for multiple injections and 10 mg/kg for a single injection. Both doses are low compared to previous ASO studies that used 10-200 mg/kg [43]. Since this approach utilized multiply tail vein injections which mainly deliver injected reagents to the liver, this approach leads to higher hepatic ASO concentrations, allowing the use of the lower ASO dose as compared to previous studies.

Another interesting aspect regarding the ASOs is their specificity within liver cell types. Thus, hnRNPU-mediated alternative splicing of TEAD1 occurs in HSCs, but not in hepatocytes or other NPC types. This finding could be explained due to alternative splicing of TEAD1 in a cell-type-specific manner [44]. Indeed, while this study shows that hnRNPU is the splicing factor that promotes Tead1 exon 5 exclusion in HSCs, other studies show that the splicing factor ESRP2 inhibits this splicing event in hepatocytes [19], and the RBFOX2 splicing factor can promote the inclusion of TEAD1 exon 5 in Hela cells [45].

Taken together these findings point to the therapeutic potential of the ASO 56 approach for the treatment of NASH. Thus, ASO 56 was specifically designed to interfere with the interaction between hnRNPU and the intronic sequences 3′ to exon 5 which are homologous between mice and humans. It is known that hnRNPU, as well as other splicing factors, can interact with the spliceosome machinery and, in some reports, this inhibits alternative splicing, [46, 47] consistent with the therapeutic strategy behind the use of ASO 56. Since it was shown that the exon5 TEAD1 splicing event that is targeted by ASO 56 is selective for HSCs and is not detected in hepatocytes or NPCs, a long-acting version of ASO 56 could have therapeutic value in a chronic disease such as NASH. Indeed, previous studies have shown that chemical modifications can be made to ASOs such that their therapeutic effects can be maintained for 3-6 months in human diseases [48, 49].

Therefore, this approach can have potential therapeutic benefits in NASH. This strategy might also apply to other disease conditions in which a therapeutic target is alternately spliced into a pathophysiologic factor.

The present studies also revealed a second mechanism by which TM7SF3 controls the Hippo pathway in HSCs. Silencing TM7SF3 induces the translocation of TAZ to the nucleus, and the effects of TM7SF3 KD on HSC activation are blocked by TAZ KD. Activation of TAZ can drive a pro-fibrotic response in vivo [50], and in recent years, studies have shown the involvement of the Hippo signaling pathway in the development of NASH and HSC activation. In hepatocytes, the role of TAZ [23, 51] in promoting fibrosis in NASH mice has been demonstrated. However, the role of TAZ in proliferation of HSCs [52] has not been previously described.

TGFβ reduced PPARγ expression in human HSCs, and TM7SF3 KD led to a further reduction in PPARγ expression levels compared to control. PPARγ is expressed in quiescent HSCs [53] and inhibits CTGF expression [54, 55]. Upon HSC activation and acquiring fibrogenic properties, the expression of PPARγ decreases [53]. Additionally, TAZ is a co-repressor of PPARγ that down-regulates PPARγ target genes, including PPARγ itself [56]. Indeed, in the present study, we show that TAZ KD in TM7SF3 KD HSCs fully prevented the TM7SF3 KD-mediated reduction in PPARγ levels.

In summary, this study uncovers a mechanism of alternative splicing regulation controlled by the nuclear protein TM7SF3. Additionally, in a preclinical model, using an ASO approach, modulation of TEAD1 alternative splicing and its activity, can reduce steatosis and fibrosis in NASH by deactivating HSCs. The importance of further work in this area is highlighted by the fact that this prevalence of NASH is rising worldwide as the leading cause of liver transplantation and hepatocellular carcinoma [57].

### Example II

Non-alcoholic fatty liver (NAFL), characterized by excessive fat accumulation in the liver with mild or no inflammation, is the most common chronic liver condition in Western populations. An estimated about 25% of patients with NAFL will develop Nonalcoholic steatohepatitis (NASH), characterized by steatosis, liver inflammation, hepatocyte damage (ballooning), and fibrosis. NASH can also lead to cirrhosis, hepatocellular carcinoma, and eventually increased liver-related mortality. Currently, there is no FDA-approved drug for NASH-induced Liver Fibrosis. In Non-alcoholic steatohepatitis (NASH) disease, quiescent Hepatic stellate cells (HSCs) undergo activation and can become proliferative, fibrogenic, contractile myofibroblasts, depositing a fibrous extracellular matrix (ECM), leading to liver scarring. As described above, a pathway was identified in which the transcription factor TEAD1 undergoes alternative splicing, which affects its activity to promote Hepatic Stellate Cell) HSC activation and liver fibrosis.

An in vivo study was conducted in which mice were placed on Non-alcoholic steatohepatitis (NASH) diet to induce fibrosis for six months. After four months of NASH diet, mice from the control group were injected with a scrambled sequence of ASO, while the treated group was injected with ASO that targeted the pre mRNA of TEAD1 and prevented the binding of hnRNP U. The outcome of this treatment for two months (a total of six months on a diet) was a reduction in Tead1 activity and deactivation of HSCs, as well as a reduction in liver fibrosis.

An antisense oligomer (ASO) was designed that specifically inhibits the binding of hnRNPU to the TEAD1 pre-mRNA. As a result, the ASO prevents alternative splicing of TEAD1 and keeps TEAD1 inactive. In in vitro studies using HSCs and in vivo studies in which the ASOs were used to treat mice on a NASH-inducing diet, it was found that the ASO robustly reduced the expression of TEAD1 alternative spliced and its downstream fibrogenic target genes. In vivo, this ameliorated the liver fibrosis typically seen in NASH.

Using ASO treatment to modulate alternative splicing may benefit the specificity of HSCs and the efficiency of deactivating HSCs. In addition, previous studies have shown that chemical modifications can be made to ASOs such that their therapeutic effects can be maintained for 3-6 months in human diseases.

Thus, ASOs can be used as a drug to reduce liver fibrosis by the deactivation of HSCs.

The ASO, e.g., specific for Hippo pathway transcription factor, TEAD1, may include one or more nucleotide analogs, e.g., having modifications to the base, e.g., nucleobases including but not limited to 1,5-dimethyluracil, 1-methyluracil, 2-amino-6-hydroxyaminopurine, 2-aminopurine, 3-methyluracil, 5-(hydroxymethyl)cytosine, 5-bromouracil, 5-carboxycytosine, 5-fluoroorotic acid, 5-fluorouracil, 5-formylcytosine, 8-azaadenine, 8-azaguanine, N⁶-hydroxyadenine, allopurinol,hypoxanthine, or thiouracil, modifications of the sugar group or modifications of the phosphate group. In one embodiment, the RNA molecule includes, but is not limited to, 1-methyladenosine, 2-methylthio-N⁶-hydroxynorvalyl carbamoyladenosine, 2-methyladenosine, 2-O-ribosylphosphate adenosine, N⁶-methyl-N-threonylcarbamoyladenosine, N⁶-acetyladenosine, N⁶-glycinylcarbamoyladenosine, N⁶-isopentenyladenosine, N⁶-methyladenosine, N⁶-threonylcarbamoyladenosine, N⁶, N⁶-dimethyladenosine, N⁶-(cis-hydroxyisopentenyl)adenosine, N⁶-hydroxynorvalylcarbamoyladenosine, 1,2-O-dimethyladenosine, N⁶,2-O-dimethyladenosine, 2-O-methyladenosine, N, N⁶,0-2-trimethyladenosine, 2-methylthio-N-(cis-hydroxyisopentenyl) adenosine, 2-methylthio-N⁶-methyladenosine, 2-methylthio-M-isopentenyladenosine, 2-methylthio-N⁶-threonyl carbamoyladenosine, 2-thiocytidine, 3-methylcytidine, N⁴-acetylcytidine, 5-formylcytidine, N⁴-methylcytidine, 5-methylcytidine, 5-hydroxymethylcytidine, lysidine, N⁴-acetyl-2-O-methylcytidine, 5-formyl-2-O-methylcytidine, 5,2-O-dimethylcytidine, 2-O-methylcytidine, N⁴,2-O-dimethylcytidine, N⁴, N⁴,2-O-trimethylcytidine, 1-methylguanosine, N²,7-dimethylguanosine, N²-methylguanosine, 2-O-ribosylphosphate guanosine, 7-methylguanosine, under modified hydroxywybutosine, 7-aminomethyl-7-deazaguanosine, 7-cyano-7-deazaguanosine, N², N²-dimethylguanosine, 4-demethylwyosine, epoxyqueuosine, hydroxywybutosine, isowyosine, N²,7,2-O-trimethylguanosine, N²,2-O-dimethylguanosine, 1,2-O-dimethylguanosine, 2-O-methylguanosine, N² N²2,2-O-trimethylguanosine, N2,N2,7-trimethylguanosine, peroxywybutosine, galactosyl-queuosine, mannosyl-queuosine, queuosine, archaeosine, wybutosine, methylwyosine, wyosine, 2-thiouridine, 3-(3-amino-3-carboxypropyl)uridine, 3-methyluridine, 4-thiouridine, 5-methyl-2-thiouridine, 5-methylaminomethyluridine, 5-carboxymethyluridine, 5-carboxymethylaminomethyluridine, 5-hydroxyuridine, 5-methyluridine, 5-taurinomethyluridine, 5-carbamoylmethyluridine, 5-(carboxyhydroxymethyl)uridine methyl ester, dihydrouridine, 5-methyldihydrouridine, 5-methylaminomethyl-2-thiouridine, 5-(carboxyhydroxymethyl)uridine, 5-(isopentenylaminomethyl)uridine, 5-(isopentenylaminomethyl)-2-thiouridine, 3,2-O-dimethyluridine, 5-carboxymethylaminomethyl-2-O-methyluridine, 5-carbamoylmethyl-2-O-methyluridine, 5-methoxycarbonylmethyl-2-O-methyluridine, 5-(isopentenylaminomethyl)-2-O-methyluridine, 5,2-O-dimethyluridine, 2-O-methyluridine, 2-thio-2-O-methyluridine, uridine 5-oxyacetic acid, 5-methoxycarbonylmethyluridine, uridine 5-oxyacetic acid methyl ester, 5-methoxyuridine, 5-aminomethyl-2-thiouridine, 5-carboxymethylaminomethyl-2-thiouridine, 5-methylaminomethyl-2-selenouridine, 5-methoxycarbonylmethyl-2-thiouridine, 5-taurinomethyl-2-thiouridine, pseudouridine, 1-methyl-3-(3-amino-3-carboxypropyl)pseudouridine, 1-methylpseudouridine, 3-methylpseudouridine, 2-O-methylpseudouridine, inosine, 1-methylinosine, 1,2-0-dimethylinosine and 2-O-methylinosine, or any combination thereof.

In one embodiment, the ASO molecule includes, but is not limited to, cytosine arabinoside or fludarabine. In one embodiment, the RNA molecule includes, but is not limited to, cladribine, acyclovir, 2′,3′-dideoxyinosine; 9-β-D-ribofuranosyladenine; .beta.-arabinofuranosylcytosine; arabinosylcytosine; 4-amino-5-fluoro-1-[(2R,5S)-2-(hydroxymethyl)-1,3-oxathiolan-5-yl]-1,2-di-hydropyrimidin-2-one; 2′,3′-dideoxy-3′-thiacytidine; 2′-3′-dideoxycytidine; {(1S,4R)-4-[2-amino-6-(cyclopropylamino)-9H-purin-9-yl]cyclopent-2-en-1-y-1}methanol: 2-Amino-9-[(1S,3R,4S)-4-hydroxy-3-(hydroxymethyl)-2-methylidenecyclopenty-1]-6,9-dihydro-3H-purin-6-one; 2′-3′-didehydro-2′-3′-dideoxythymidine: 1-(2-deoxy-.beta.-L-erythro-pentofuranosyl)-5-methylpyrimidine-2,4(1H,3H)-dione; 1-[(2R,4S,5S)-4-azido-5-(hydroxymethyl)oxolan-2-yl]-5-methylpyrimi-dine-2,4-dione; 1-[(2R,4S,5R)-4-hydroxy-5-(hydroxymethyl)oxolan-2-yl]-5-iodo-1,2,3,4-tetr-ahydropyrimidine-2,4-dione; 1-[4-hydroxy-5-(hydroxymethyl)oxolan-2-yl]-5-(trifluoromethyl) pyrimidine-2,4-dione; 5-Fluoro-2′-deoxycytidine; 5-Fluorodeoxycytidine; Floxuridine (5-Fluoro-1-[4-hydroxy-5-(hydroxymethyl)tetrahydrofuran-2-yl]-1H-pyrimidi-ne-2,4-dione); 4-amino-1-(2-deoxy-2,2-difluoro-β-D-erythro-pentofuranosyl)pyrimidin-2(1H)-one; or 2′,2′-difluoro-2′-deoxycytidine; (8R)-3-(2-deoxy-β-D-erythro-pentofuranosyl)-3,4,7,8-tetrahydroimidaz-o[4,5-d][1,3]diazepin-8-ol, or any combination thereof.

In one embodiment, a strand of the RNA may include analogs such as 2′-O-methyl-substituted RNA, locked nucleic acid (LNA) or BNA (Bridged Nucleic Acid), morpholino, or peptide nucleic acid (PNA), or any combination thereof.

In one embodiment, nucleotide analogs include phosphorothioate nucleotides or deazapurine nucleotides and other nucleotide analogs.

In one embodiment, the ASO molecule can independently include a modified nucleotide selected from a deoxyribonucleotide, a dideoxyribonucleotide, an acyclonucleotide, a 3′-deoxyadenosine (cordycepin), a 3′-azido-3′-deoxythymidine (AZT), a 2′,3′-dideoxyinosine (ddl), a 2′,3′-dideoxy-3′-thiacytidine (3TC), a 2′,3′-didehydro-2′,3′-dideoxythymidine (d4T), a monophosphate nucleotide of 3′-azido-3′-deoxythymidine (AZT), a 2′,3′-dideoxy-3′-thiacytidine (3TC) and a monophosphate nucleotide of 2′,3′-didehydro-2′,3′-dideoxythymidine (d4T), a 4-thiouracil, a 5-bromouracil, a 5-iodouracil, a 5-(3-aminoallyl)-uracil, a 2′-O-alkyl ribonucleotide, a 2′-O-methyl ribonucleotide, a 2′-amino ribonucleotide, a 2′-fluoro ribonucleotide, or a locked nucleic acid; or any combination thereof.

In one embodiment, the nucleotide modification includes 2′ modifications, e.g., 2′ F on pyrimidines or 2′ H or 2′ OMe on purines.

In one embodiment, the nucleotide modification includes a phosphate backbone modification selected from a phosphonate, a phosphorothioate, a phosphotriester; a morpholino nucleic acid; or a peptide nucleic acid (PNA).

Sugar modifications include, but are not limited to, replacing the heteroatoms at the 2′ and 3′ carbons with hydrogen, another heteroatom or an alkyl group; replacing the H's at the 2′ carbon with a heteroatom or alkyl group; replacing the 2′ and 3′ carbons with a heteroatom, most commonly S or O; removing the 2′ and/or 3′ carbons to generate acyclic sugars; replacing the 4′-OH with N, S, or an alkyl group; adding alkyl groups to the 4′-carbon; replacing the 5′-hydroxyl with N or a phosphonate, or interconversion of both the sugar stereochemistry (D vs. L) and anomeric configuration (α vs. β).

The ASO inhibits expression of, for example, TEAD1 having the following amino acid sequence (SEQ. ID. NO: 3)

MEPSSWSGSESPAENMERMSDSADKPIDNDAEGVWSPDIEQSFQ
EALAIYPPCGRRKIILSDEGKMYGRNELIARYIKLRTGKTRTRKQVSS
HIQVLARRKSRDFHSKLKDQTAKDKALQHMAAMSSAQIVSATAIHNKL
GLPGIPRPTFPGAPGFWPGMIQTGQPGSSQDVKPFVQQAYPIQPAVTA
PIPGFEPASAPAPSVPAWQGRSIGTTKLRLVEFSAFLEQQRDPDSYNK
HLFVHIGHANHSYSDPLLESVDIRQIYDKFPEKKGGLKELFGKGPQNA
FFLVKFWADLNCNIQDDAGAFYGVTSQYESSENMTVTCSTKVCSFGKQ
VVEKVETEYARFENGRFVYRINRSPMCEYMINFIHKLKHLPEKYMMNS
VLENFTILLVVTNRDTQETLLCMACVFEVSNSEHGAQHHIYRLVKD

TEAD1 having the amino acid sequence of SEQ ID NO: 3 has the following nucleic acid sequence (SEQ ID NO. 2):

(SEQ ID NO: 2)
attccgaaca ttcttagcat cgctcgcgcc gcgccgcgcc gcctgagccg agccgagcct
ctgctgccgc cgccgcggcc ccgccgcccg ccgcgggcgc ccaccaagca ctttgcagac
tcgcttccac cctgcgggcc attccgcgcg gcggggcccg ggcccggggc ggccgcgtcc
aggcacaggc catgcagtga cgccccccca cccctccacc tttgcccgga gcgcgggcag
cagcccagcg cgccagccgg ccccggggca ggagcggtgc taggcagggg tggggtggcc
gggcccaggg accgggagcc ggggagggag ccgggcaccg agcagagggc gggggaagcg
gcgccgaagt ttgcctcgga ctcgccgggc gctgcggtgg ctccctgggc cgaggactgt
tgctgccgct gccgccgccg cttcattgca cattcaagtg gaaaattttc aggagtcagc
agaaacattg tgtccaaaaa agactgagtc gcagttacca ccaaacccag gaggagactc
tccctggaaa acttcccttc cctttcggtt tattttcttg aaaaggctcc aggcttcggc
ttggaaaatc ccaccgccaa aattgagccc agcagctgga gcggcagtga gagccctgcc
gaaaacatgg aaaggatgag tgactctgca gataagccaa ttgacaatga tgcagaaggg
gtctggagcc ccgacatcga gcaaagcttt caggaggccc tggctatcta tccaccatgt
gggaggagga aaatcatctt atcagacgaa ggcaaaatgt atggtaggaa tgaattgata
gccagataca tcaaactcag gacaggcaag acgaggacca gaaaacaggt gtctagtcac
attcaggttc ttgccagaag gaaatctcgt gattttcatt ccaagctaaa ggatcagact
gcaaaggata aggccctgca gcacatggcg gccatgtcct cagcccagat cgtctcggcc
actgccattc ataacaagct ggggctgcct gggattccac gcccgacctt cccaggggcg
ccggggttct ggccgggaat gattcaaaca gggcagccag gatcctcaca agacgtcaag
ccttttgtgc agcaggccta ccccatccag ccagcggtca cagcccccat tccagggttt
gagcctgcat cggccccagc tccctcagtc cctgcctggc aaggtcgctc cattggcaca
accaagcttc gcctggtgga attttcagct tttctcgagc agcagcgaga cccagactcg
tacaacaaac acctcttcgt gcacattggg catgccaacc attcttacag tgacccattg
cttgaatcag tggacattcg tcagatttat gacaaatttc ctgaaaagaa aggtggctta
aaggaactgt ttggaaaggg ccctcaaaat gccttcttcc tcgtaaaatt ctgggctgat
ttaaactgca atattcaaga tgatgctggg gctttttatg gtgtaaccag tcagtacgag
agttctgaaa atatgacagt cacctgttcc accaaagttt gctcctttgg gaagcaagta
gtagaaaaag tagagacgga gtatgcaagg tttgagaatg gccgatttgt ataccgaata
aaccgctccc caatgtgtga atatatgatc aacttcatcc acaagctcaa acacttacca
gagaaatata tgatgaacag tgttttggaa aacttcacaa ttttattggt ggtaacaaac
agggatacac aagaaactct actctgcatg gcctgtgtgt ttgaagtttc aaatagtgaa
cacggagcac aacatcatat ttacaggctt gtaaaggact gaacatggtt atttatatat
atagatatct gtatatacac acacacatat gtgcacacac acactctctc tccattatcg
aacgactgac tgtaaacctc accacacagg gtggtgccct ggccccgagg tcaccccgac
ttttctaaat cttgtttgag tgaagtcatt ttttcatgtg ttcatactat cattgtagct
gtgaagttct ggtacagttg taaaaagaga aattgagttg tttctctatg ttcttcagat
gtgcagccca caattcctcg ggaaaggtga acctgaacaa cccaagtctc tctctgcaga
gccctgtttc taattgtggt agaaaatatt gagacagagc atttgccatg ggacatttac
agcctttata caaatgtatt tagttctctt ttttccaaca taaaattctt gttttaagat
acaagtaaaa ttaatcttta aatataaatg taaattagta cacaaaacta agaatcttta
gacttatctt tgtaactaat tagggtggaa gttatgaaag aatgtaattc actaaattat
tttttaaatg aaaccttttt ttttcttttt gaaaccaaat gttaaactat agccttaaga
aatgcttggt agaagtgtcc taatgagaca aatttgtact tttatcctca aggttaacac
taatctccta atccattaaa ctcttgaaca ggtattacaa aggaagaaaa cttcacccct
tatccttaac atatatagta tatttaaaaa atataaaatt gtattgtact aatgtgatga
tggattattt aatgaaaaag aaaaaatggc tctttttgca ataagtagat acatactgaa
aaaatctaaa cttacaatgt ttatagtctt gtgtgtgcag ttatatttta tatggacgac
caaatttttt attaagatga gtaaatattt gaaccactga attttaataa caaaatttta
aaattggcat gaatacggaa tactgcactg tgagatgcaa agtatacaga atctgtggct
gggagaaaat ttcatcaaat agacaagtaa aaggctcatc agttttagca tctctgctcc
ccagaaaatt gtaagcatcc tcaccagcct gtggatacat tctttatttc tagtgaccca
atatgcatat taacctgcta taactagggc tatatgtgta ggtatgtgta tacatataca
caaatgcaca tatagagtta acacatttag tgaacacttg tttagtgtca ctcagtttgc
taggtgctga tatgtacgta tatctcaatg tgtctgtaga cttagataca tcctcttgaa
gcacatccat ttctttagcg tctctcagta agttacagta cttgtttgac ttaggtttaa
gaggcccagc tacctatctc tgaccttttc aaataggctc atttgggaga ttcttttgcc
aggagagatt caactttcca atctaagtat tccagagcat tgcccaggca gagttggttt
gatgtggcca gatgttttga gttatttccc ttaagtgttt cactggggag agaacaggga
gtgctcctcc agcttcccaa agaaatatgt ttttgtaagt ggtaggaaca tgtgcacaca
atagaacatg aaataagttt tttaacttgt aaaacatgtc aagatttttc caccaagcta
gaaaataaaa aacttagttc taccacatcc aattaactta cacaccccct tccctgtctc
aacacctgct ttgaccctgc ttttctatta ttacatcagt cagcatcttg tggtccctaa
catgaggatg tggctggctc gtgggaaaca gcaaaacact aagcctgacc tctcccaaat
tgggaagacc agaggagaaa gtgcaaaact gtccccattt ggaatgccca ttccttctag
aaaccagttg gacagtgctc ctctgccctt cataaacaga ctactgttgg gtccctgatt
ccaggctggc ctgtgaagga ttgccccagg tgtccccttt cacggttgtc acatttacag
tgacttctgt tgaacacccc tcttagggat gtttcttttg ctcttatttc ctgcatcttt
ccttaaggga agccccatcc tctcccagga ccaggagttt atgaccaggc gagcacaaat
ggctaaaagc caagctgtcc tagaacttca gtgggagagc tgtctggttc atattctacc
caggaatggt acttttcagt gcagccagga gggctcttgg gatttccttt ccaaagcaca
aaaatactgg gacccaagaa gaacagctag aggacaactc tcttggcaca gagacgggga
cagcccagtc tgctgacctc acagggtcag ctgggccccc ctggtgcttc accacctgca
tcctcttgct cagaatgcct ttgcagttga gttttctggg tttctatgat tgaccttgag
gtttactcct tgctcttaca acatttctaa ggatttttaa aagtttactt cttgtcttgt
tcttctaaag ctttctccag gacagatatt ttccctgtct taaccactgg tccagtcatc
ccagtgggct tctctttgtc tctcccagat tagacctttg ggtgagattg gcatcacaac
atctaatctg agtctgtctt ttgtccttca ttctgtatgg cagtctccct ttgttataaa
agctttctaa agcatactaa agaagccttc ccagagcccc gtcttgcttc tcttccaggt
gctctatccc ctcgagaccc tctggtgcca ggcttgcttc acggccatct tgtgttgtca
ctgcagagtt tggaggccag ttttccacag cctaaacagg gaggagctgc agaatggggc
tctggtctct gggcattcat ttccctcata gaggctgaga ataaaacaag gacttattca
cacatgttct agaaccccag aatggcccaa gttacctgag accagggttt ctcaaccttg
acaccattga cattttggac tgggtaattc tttgttctgc agagctgtcc tttgcactgt
aggagattta ctaatatccc tggcctctac ccagtagtac cactagcacc tattccccac
ccagcgtgtc tccagatatt gtcaaatatc ccatcgggtg caaaatgatc cctggtcaag
atctgttgcc caagatgtta caggtcacaa tgaccacatt tgaaattgtt ttccctttca
ttttaccctg tgaaagcatc tctcctagag ccttgcaaga ggcaggtgac attgtgtcca
tatttcttcc tgtttcagaa cttctgtttc acaacaattt ctctctcgct acaagtattc
tttcactcag cactggggaa gttgggaaca gctggtcacc atcatccctt taatcaactc
acacctgttt aaagagtgtt tctgatttga ccttcatccc ttagtttact ggcgttaaaa
aaagtctcag caattttcat tatttctcgt gggtctcatt atcaaacctt tacttatttc
ggcatatttc ctctgggctt cttctagttt ctgccttaca agcaatgctg ttctgtaaat
ttattgaaac ctctggaaca tttcaccttt agagatggag gatggaagga ttggtaccag
aagagggcta agatacgttt tctgtcttga gctgaaagca cagtctactc tccttcgttt
tgtcgatgag aaagttgagg ccagagggga ggtgacatgt ttagagtcac ccagctggtt
agtgacagaa aaagcgtgag agttgtctag gattcctgcc actttggtcc ctggcctctc
ctgggggagg ctgctgttct taggtgctct aagcttaatc cctcagaatg tgtggacagg
tcagcttaga agagatgggg agattcagga tccccctgtg ccagagcaca gcctcaccgg
atgctgcttc ccacactgaa gtgtcctgtc cgaccattgc tatctgaggc atccacaagc
aggtaggaaa gctggcgagc cattttactt cctgaggaca attccccagc cacaggctct
gagtcaaatt tctatttggt aagcatccta gcagcaaagt cctgcactca gaccagccaa
aaaacagccc ccattccaag tacttggtgt caaaagtccc cgaacgactt ttaaacccaa
gtcttcttaa ggtttcagta ctgtggtggc tttagcagtt gtttttgtgc aactataaat
tatttaaatc atctgagatg acagtcaatt ttacaaacca ggtacatatt aatttgtata
attttgtata tgctctggta cactacctga actaacgaag ggtagaacta attctgtttg
tcagtgttca cacctgtaac attaggagga tatgtctgca ttgcttattt ctttatgttg
gtgtttctgt ggcaaagccc tgcacatggc atttctgaaa agccttaaat ctttaagatg
ttgcatgtag ggtatgcagt gcaaaaggct gcctcagaac tgtgagccct tttgtaagct
ggaagcattt ctcttactac tgttactttt gtaggaagtt ttcaattcag agctgccaaa
gtgttcccgt aagcagtgcc ttagtaatac cttagtcatg ccgccagcct tttcttacac
caattcctaa tgttcattta cgaattggcc caatattgga aacaaaacaa gcaaaaattg
tcttcatttt tgttttgtaa gcccattttt tctccagttc tataggaaac tgactgcttg
gtgtaaaatc cgaaactgga cacaagtcag ttctttcacc acactcaaat gtatatacca
aaacaaaagg ttgcaacttc atagtttact atgaaaagca aattgtactt tttaatgttg
ccttttaaat tcatgaccaa atacttagct atttgtgaat cttctgcact ctagcatgaa
agtgcctttg gtttgagatt ccagcttaga aaagtgctgc cataataacg ataatttgta
gagagaccaa aaatattttg agatcaccgt aatgcctttg gtttaccggg atgagtaacc
aaccacaggc ctctgttcac aagagcacga cgtggtcccc gcctgctgct agtctgtctg
ccactggggg cctcccaaca tccatagcac acttcagcgg aaggacccca gaaactgttg
tgtttgtgtg tgctgatgac ctagtgtgtc atttcacctc gtcacccagc cctgcgtccg
gatgagggga cttctgcaca aatgacagaa tctcggctgg tggacagata ctacagcttt
ctcctcctcc ttgtgttcgt gttcagtctc tgtggagact ttcttttcca ttcaaatgac
agtgcgcact tatctggttt acacaatgat accattttga aagttggaag cctcaaactg
agacgacagt gcagaacaaa acaaaagtga gttagggtcg ttaaaattga agtgttcttc
ttagggcaaa catgttgact ccgagtattg tgtatgaatg tgctacgaga aacttccaaa
gagcaccatt cacaatttgg cattttcaaa gaatgttcca gccctcaaag gggcaactct
ttaaagtcct tcttggcttt tatccaaacc ttgtagaaat tgggaaagct gatagaggta
aggaagacga gtgaaaagga caagaaggcc aaacaccagc caaaaagaaa ctaggaaaaa
aagattttct ttgctaatat agatgtaaaa ataacatcag acatctttga aaattagcct
ctaaactctt aatacatacg ttctgtgtgt ctctacctgg cgtctttaag aatatcctct
ctgggctctg aaattttagg agtgattctt atccactcca agttgtaagt atttgtagaa
atttgtgcaa acaaacaaaa actatcaaat gaaaagaaaa tgtactcaac ctaacttata
gttagcagct ggaattctca actcttccct gccagcacta taccacagtg tggaagaaat
tagtcaaatg cttgttttcc tgcttctctt ttcaactgtt actgtgcttt gtttgaaagt
agttttctct ctcaaagccg ttgcttatat cgttaagaat gaaggtttgt gtttaaaatt
tattgcattg caaagggtag tttcactgaa gtcatgcacc attaaataag atgaaatatt
tgtatttatt gtcctacttc ctaagccgta acttcttttc ctctgtgaat ttgcattgag
tcactcatgc tacactacat cgctttagta tttgagatgg catttatgtt tcctctcgtt
tatcatgaaa tggggtcaga ttccatcaga ttccacctct gtcaggtgga ctcttgtctg
ccttccatga tgagattttt tttctccttc ccctttcttt aagagaggct gacagatcta
ggtgtcaatc aattggaaac cagtctctga ttttttttca ttagttattt tctatcatta
gtttcactgt gtaaattaga tatcaactgc acttctttaa aaaaaaatac atctccctat
tacctccttg aaagatttac ttctgtaggc ctttttcaat aggctcatga ctgcagacaa
ggaaaaaaaa agtaaaaaca aaaacagtat gtgcctgaaa atgacaaaaa aaaaatttgt
aacatttaaa aaagaaacct gaatagcctt taattcttta ataatacact taaattttat
gtaaatcggt tttcgccacg tgtgtttgtt cacattctaa atgacttaat gggattctca
cggtctgtgt ctttgtgtca cgtgtataaa atgggcttgt gatgtaagcg tttcatctgg
tcagtggttc ctttgatatt gtactgctgc tgggagtggg ctgtggaacc tgccttcggg
taactgggtt cctcttgggt agattggaga gatgggggtg ggcgtgggca aattctcaca
catgttttct taacctattt gcagaaactt tcaaaaggca tttgattaaa cctcttggca
gtacagtatt cttgtatttg ttaacgtctg tgtttaggta ctggtacctt tttgttttaa
aatgttctaa gtgttggctt taaagtgaat ttatctttag tatgatagtt atatgaaaat
tataggattt gtgtgcagag aattttttta taaagtgctt tgtaaaaaaa aaaaaatgta
ttctagcttt tgcggtacat atgtgtgata actttaatac ccatgacagt taagtgcaat
tatttcatca ctctaaaaat gctatttttg tgtcagttcc tgcaggtgtt ttcatgtctt
tgcaaagtga cacattttga tgccttcttg ataaagtggt agacattttg tagctttcta
gaaactttgt attcatacgg tatcaatgaa aaataaagaa aatgaaagtg tgggtca

An example ASO is: 5′-CAGCUAAAAUACAGAAUACUCA-3′ (SEQ ID NO:1). In addition, this RNA molecule may have two modifications within each gap between two nucleotides.
1. 2′-MethoxyEthoxy (2MOE)
2. Phosphorothioate Bond (*)

ASO 56:
/5′-2MOE-C/*/i2MOE-A/*/i2MOE-G/*/i2MOE-C/*/i2MO E-U/*/i2MOE-A/*/i2MOE-A/*/i2MOE-A/*/i2MOE-A/*/i 2MOE-U/*/i2MOE-A/*/i2M OE-C/*/2MOE-A/*/i2MOE-G/*/i2MOE-A/*/i2MOE-A/*/i2MOE-U/*/i2MOE-A/*/i2 MOE-C/*/i2MOE-U/*/i2MOE-C/*/3′-2MOE-A/

### Example III

Targeting TEAD1 Pre-mRNA with ASO 56 Causes HSC Deactivation and Decreased Liver Fibrosis in Primary Sclerosing Cholangitis (PSC)

Primary Sclerosing Cholangitis (PSC) is a chronic liver disease characterized by inflammation and fibrosis of the bile ducts. ASO 56, a targeted antisense oligomer having a sequence identical for mouse and humans, has demonstrated remarkable efficacy in reducing liver fibrosis associated with PSC, offering a promising therapeutic approach for this debilitating disease.

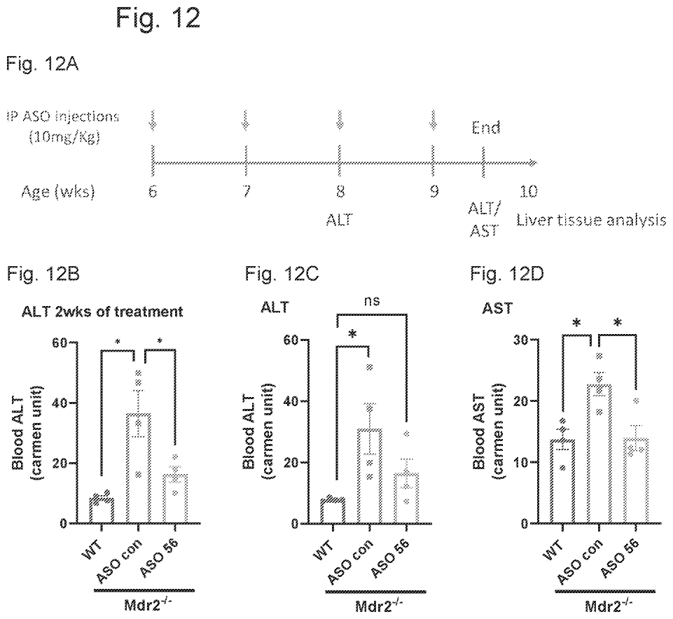

The Mdr2 KO mouse model is a relevant model for PSC which develops severe fibrotic liver disease by 12 weeks of age. In these mice, ASO treatment was initiated at 6 weeks old. Weekly intraperitoneal injections of ASO 56 were administered for nearly four weeks (FIG. 12A). The study cohort included wild-type mice, Mdr2 KO mice treated with a control ASO, and Mdr2 KO mice treated with ASO 56.

Blood levels of alanine aminotransferase (ALT) and aspartate aminotransferase (AST) were measured at two weeks and at the end of the experiment, and showed a significant reduction in the ASO 56-treated group compared to the ASO control group (FIGS. 12B, C, D).

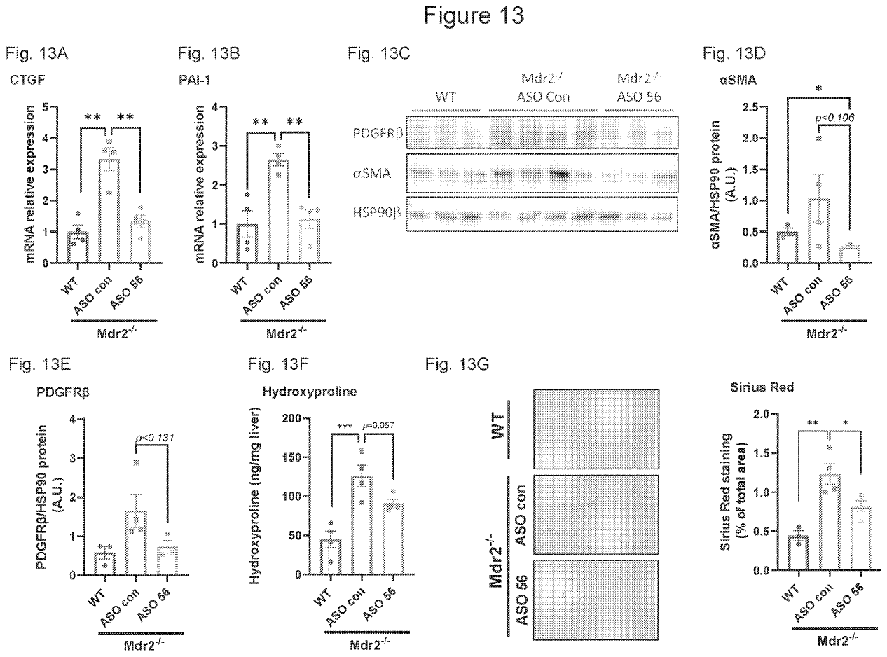

In the liver tissues, ASO 56 treatment led to a significant reduction in the expression of the fibrogenic genes, CTGF and PAI-1 (FIG. 17A), in the liver. Additionally, ASO 56 treatment resulted in decreased expression of PDGFRb (FIG. 17C) and αSMA (FIG. 13D) proteins compared to the ASO control group.

Furthermore, liver hydroxyproline content, a marker of fibrosis, was 30% lower in the ASO 56-treated group compared to the ASO control group. At the same time, control Mdr2KO mice exhibited a 2.5-fold increase in hydroxyproline content compared to WT (FIG. 13E).

These results highlight the efficacy of ASO 56 in reducing HSC activation and liver fibrosis in the Mdr2 KO mouse model, suggesting its therapeutic potential in fibrotic liver diseases such as PSC.

### Statements

1. A method to prevent, inhibit or treat liver disease in a mammal, comprising:
administering to the mammal a composition comprising an effective amount of an antisense oligonucleotide specific for human Hippo pathway transcription factor, TEAD1.

2. The method of statement 1, wherein the mammal is a human.

3. The method of statement 1 or 2, wherein the composition is systemically administered.

4. The method of statement 1 or 2, wherein the composition is orally administered.

5. The method of any one of statements 1 to 4, wherein the composition is injected.

6. The method of any one of statements 1 to 5, wherein the antisense oligonucleotide has at least 80%, 82%, 84%, 85%, 87%, 90%, 92%, 94%, 95%, 97%, 98% or 99% nucleotide sequence identity to ASO 56.

7. The method of any one of statements 1 to 6, wherein the antisense oligonucleotide has at least one or more nucleotide analogs.

8. The method of any one of statements 1 to 7, wherein the mammal has or is at risk of having liver fibrosis

9. The method of any one of statements 1 to 8, wherein the mammal has or is at risk of having Primary Sclerosing Cholangitis (PSC) or Non-alcoholic steatohepatitis (NASH).

10. A method to prevent, inhibit or treat fibrosis in a mammal, comprising: administering to the mammal a composition comprising an effective amount of an antisense oligonucleotide specific for human Hippo pathway transcription factor, TEAD1.

11. The method of statement 10, wherein the mammal is a human.

12. The method of statements 10 or 11, wherein the composition is systemically administered.

13. The method of statements 10 or 11, wherein the composition is orally administered.

14. The method of any one of statements 10 to 13, wherein the composition is injected.

15. The method of statements 10 or 14, wherein the composition is locally administered.

16. The method of any one of statements 10 to 15, wherein the antisense oligonucleotide has at least 80%, 82%, 84%, 85%, 87%, 90%, 92%, 94%, 95%, 97%, 98% or 99% nucleotide sequence identity to ASO 56.

17. The method of any one of statements 10 to 16, wherein the antisense oligonucleotide has at least one or more nucleotide analogs.

18. The method of any one of statements 10 to 17, wherein the mammal has or is at risk of having liver fibrosis.

19. The method of any one of statement 1 to 18, wherein the composition comprises liposomes.

20. The method of any one of statements 1 to 19, wherein the composition comprises nanoparticles.

21. A method to prevent, inhibit or treat cholangitis in a mammal, comprising:
administering to the mammal a composition comprising an effective amount of an antisense oligonucleotide specific for human Hippo pathway transcription factor, TEAD1.

22. The method of statement 21, wherein the mammal is a human.

23. The method of statements 21 or 22, wherein the composition is systemically administered.

24. The method of statements 21 or 22, wherein the composition is orally administered.

25. The method of any one of statements 21 to 24, wherein the composition is injected.

26. The method of statements 21 or 25, wherein the composition is locally administered.

27. The method of any one of statements 21 to 26, wherein the antisense oligonucleotide has at least 80%, 82%, 84%, 85%, 87%, 90%, 92%, 94%, 95%, 97%, 98% or 99% nucleotide sequence identity to ASO 56.

28. The method of any one of statements 21 to 27, wherein the antisense oligonucleotide has at least one or more nucleotide analogs.

29. The method of any one of statements 21 to 28, wherein the composition comprises liposomes.

30. The method of any one of statements 21 to 29, wherein the composition comprises nanoparticles.

31. A composition comprising an antisense oligonucleotide specific for human Hippo pathway transcription factor, TEAD1.

32. The composition of statement 31, wherein the wherein the antisense oligonucleotide has at least 80%, 82%, 84%, 85%, 87%, 90%, 92%, 94%, 95%, 97%, 98% or 99% nucleotide sequence identity to ASO 56.

The specific methods, devices and compositions described herein are representative of example embodiments and not intended as limitations on the scope of the technology. Other objects, aspects, and embodiments will occur to those skilled in the art upon consideration of this specification, and are encompassed within the spirit of the technology as defined by the scope of the claims. It will be readily apparent to one skilled in the art that varying substitutions and modifications can be made to the technology disclosed herein without departing from the scope and spirit of the technology.

The technology illustratively described herein suitably can be practiced in the absence of any element or elements, or limitation or limitations, which is not specifically disclosed herein as essential. The methods and processes illustratively described herein suitably can be practiced in differing orders of steps, and the methods and processes are not necessarily restricted to the orders of steps indicated herein or in the claims.

Under no circumstances can the patent be interpreted to be limited to the specific examples or embodiments or methods specifically disclosed herein. Under no circumstances can the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the technology as claimed. Thus, it will be understood that although the present technology has been specifically disclosed by example embodiments and optional features, modification and variation of the concepts herein disclosed can be resorted to by those skilled in the art, and that such modifications and variations are considered to be within the scope of this technology as defined by the appended claims and statements of the technology.

The technology has been described broadly and generically herein. Each of the narrower species and subgeneric groupings falling within the generic disclosure also form part of the technology. This includes the generic description of the technology with a proviso or negative limitation removing any subject matter from the genus, regardless of whether or not the excised material is specifically recited herein. In addition, where features or aspects of the technology are described in terms of Markush groups, those skilled in the art will recognize that the technology is also thereby described in terms of any individual member or subgroup of members of the Markush group.

### REFERENCES

1. Benedict, M.& X. Zhang, (2017). Non-alcoholic fatty liver disease: An expanded review. World J Hepatol. 9, (16), 715-732.
2. Vieira Barbosa, J.& M. Lai, (2021). Nonalcoholic Fatty Liver Disease Screening in Type 2 Diabetes Mellitus Patients in the Primary Care Setting. Hepatol Commun. 5, (2), 158-167.
3. Godoy-Matos, A. F., W. S. Silva Junior, & C. M. Valerio, (2020). NAFLD as a continuum: from obesity to metabolic syndrome and diabetes. Diabetol Metab Syndr. 12, 60.
4. Zisser, A., D. H. Ipsen, & P. Tveden-Nyborg, (2021). Hepatic Stellate Cell Activation and Inactivation in NASH-Fibrosis-Roles as Putative Treatment Targets?Biomedicines. 9, (4).
5. Hou, W.& W. K. Syn, (2018). Role of Metabolism in Hepatic Stellate Cell Activation and Fibrogenesis. Front Cell Dev Biol. 6, 150.
6. Tomita, K., et al., (2014). Free cholesterol accumulation in hepatic stellate cells: mechanism of liver fibrosis aggravation in nonalcoholic steatohepatitis in mice. Hepatology. 59, (1), 154-169.
7. Kim, C. L., S. H. Choi, & J. S. Mo, (2019). Role of the Hippo Pathway in Fibrosis and Cancer. Cells. 8, (5).
8. Moroishi, T., et al., (2015). A YAP/TAZ-induced feedback mechanism regulates Hippo pathway homeostasis. Genes Dev. 29, (12), 1271-1284.
9. Boopathy, G. T. K.& W. Hong, (2019). Role of Hippo Pathway-YAP/TAZ Signaling in Angiogenesis. Front Cell Dev Biol. 7, 49.
10. Zhang, K., et al., (2016). omega-3 PUFAs ameliorate liver fibrosis and inhibit hepatic stellate cells proliferation and activation by promoting YAP/TAZ degradation. Sci Rep. 6, 30029.
11. Mannaerts, I., et al., (2015). The Hippo pathway effector YAP controls mouse hepatic stellate cell activation. J Hepatol. 63, (3), 679-688.
12. Du, K., et al., (2018). Hedgehog-YAP Signaling Pathway Regulates Glutaminolysis to Control Activation of Hepatic Stellate Cells.

Gastroenterology. 154, (5), 1465-1479 e1413.
13. Isaac, R., et al., (2017). TM7SF3, a novel p53-regulated homeostatic factor, attenuates cellular stress and the subsequent induction of the unfolded protein response. Cell Death Differ. 24, (1), 132-143.
14. Isaac, R., Vinik, Y., Mikl, M., Shatz-Azoulay, H., et al., (2022). A Seven-Transmembrane Protein-TM7SF3, Resides in Nuclear Speckles and Regulates Alternative Splicing. Accepted for publication, iScience
15. Vassilatis, D. K., et al., (2003). The G protein-coupled receptor repertoires of human and mouse. Proc Natl Acad Sci USA. 100, (8), 4903-4908.
16. Harrison, S. P., S. F. Baumgarten, R. Verma, O. Lunov, A. Dejneka, & G. J.

Sullivan, (2021). Liver Organoids: Recent Developments, Limitations and Potential. Front Med (Lausanne). 8, 574047.
17. Gao, H., et al., (2022). MiR-690 treatment causes decreased fibrosis and steatosis and restores specific Kupffer cell functions in NASH. Cell Metab. 34, (7), 978-990 e974.
18. Gandhi, C. R., (2017). Hepatic stellate cell activation and pro-fibrogenic signals. J Hepatol. 67, (5), 1104-1105.
19. Bangru, S., et al., (2018). Alternative splicing rewires Hippo signaling pathway in hepatocytes to promote liver regeneration. Nat Struct Mol Biol. 25, (10), 928-939.
20. Liu, X., et al., (2020). Identification of Lineage-Specific Transcription Factors That Prevent Activation of Hepatic Stellate Cells and Promote Fibrosis Resolution. Gastroenterology. 158, (6), 1728-1744 e1714.
21. Stein, C., et al., (2015). YAP1 Exerts Its Transcriptional Control via TEAD-Mediated Activation of Enhancers. PLoS Genet. 11, (8), e1005465.
22. Tao, L., et al., (2018). MicroRNA-942 mediates hepatic stellate cell activation by regulating BAMBI expression in human liver fibrosis. Arch Toxicol. 92, (9), 2935-2946.
23. Wang, X., et al., (2016). Hepatocyte TAZ/WWTR1 Promotes Inflammation and Fibrosis in Nonalcoholic Steatohepatitis. Cell Metab. 24, (6), 848-862.
24. Lee, Y.& D. C. Rio, (2015). Mechanisms and Regulation of Alternative Pre-mRNA Splicing. Annu Rev Biochem. 84, 291-323.
25. Prakash, T. P., et al., (2014). Targeted delivery of antisense oligonucleotides to hepatocytes using triantennary N-acetyl galactosamine improves potency 10-fold in mice. Nucleic Acids Res. 42, (13), 8796-8807.
26. Zhang, L. P., et al., (1999). Increased expression of plasminogen activator and plasminogen activator inhibitor during liver fibrogenesis of rats: role of stellate cells. J Hepatol. 31, (4), 703-711.
27. Salguero Palacios, R., et al., (2008). Activation of hepatic stellate cells is associated with cytokine expression in thioacetamide-induced hepatic fibrosis in mice. Lab Invest. 88, (11), 1192-1203.
28. Tan, Z., et al., (2013). IL-17A plays a critical role in the pathogenesis of liver fibrosis through hepatic stellate cell activation. J Immunol. 191, (4), 1835-1844.
29. Yosef, R., et al., (2017). p21 maintains senescent cell viability under persistent DNA damage response by restraining JNK and caspase signaling. EMBO J. 36, (15), 2280-2295.
30. Lin, K. C., H. W. Park, & K. L. Guan, (2017). Regulation of the Hippo Pathway Transcription Factor TEAD. Trends Biochem Sci. 42, (11), 862-872.
31. Liu, C. Y., et al., (2010). The hippo tumor pathway promotes TAZ degradation by phosphorylating a phosphodegron and recruiting the SCF{beta}-TrCP E3 ligase. J Biol Chem. 285, (48), 37159-37169.
32. Hiemer, S. E., A. D. Szymaniak, & X. Varelas, (2014). The transcriptional regulators TAZ and YAP direct transforming growth factor beta-induced tumorigenic phenotypes in breast cancer cells. J Biol Chem. 289, (19), 13461-13474.
33. Liu, Y., et al., (2013). Transforming growth factor-beta (TGF-beta)-mediated connective tissue growth factor (CTGF) expression in hepatic stellate cells requires Stat3 signaling activation. J Biol Chem. 288, (42), 30708-30719.
34. De Smet, V., et al., (2021). Initiation of hepatic stellate cell activation extends into chronic liver disease. Cell Death Dis. 12, (12), 1110.
35. Lai, D., K. C. Ho, Y. Hao, & X. Yang, (2011). Taxol resistance in breast cancer cells is mediated by the hippo pathway component TAZ and its downstream transcriptional targets Cyr6l and CTGF. Cancer Res. 71, (7), 2728-2738.
36. Norman, M., C. Rivers, Y. B. Lee, J. Idris, & J. Uney, (2016). The increasing diversity of functions attributed to the SAFB family of RNA-/DNA-binding proteins. Biochem J. 473, (23), 4271-4288.
37. Xiong, J., et al., (2020). hnRNPU/TrkB Defines a Chromatin Accessibility Checkpoint for Liver Injury and Nonalcoholic Steatohepatitis Pathogenesis. Hepatology. 71, (4), 1228-1246.
38. Gupta, M. P., P. Kogut, & M. Gupta, (2000). Protein kinase-A dependent phosphorylation of transcription enhancer factor-1 represses its DNA-binding activity but enhances its gene activation ability. Nucleic Acids Res. 28, (16), 3168-3177.
39. Jiang, S. W., M. Dong, M. A. Trujillo, L. J. Miller, & N. L. Eberhardt, (2001). DNA binding of TEA/ATTS domain factors is regulated by protein kinase C phosphorylation in human choriocarcinoma cells. J Biol Chem. 276, (26), 23464-23470.
40. Sivanesan, S., M. D. Howell, C. J. Didonato, & R. N. Singh, (2013). Antisense oligonucleotide mediated therapy of spinal muscular atrophy. Transl Neurosci. 4, (1).
41. Geary, R. S., D. Norris, R. Yu, & C. F. Bennett, (2015). Pharmacokinetics, biodistribution and cell uptake of antisense oligonucleotides. Adv Drug Deliv Rev. 87, 46-51.
42. Graham, M. J., et al., (1998). In vivo distribution and metabolism of a phosphorothioate oligonucleotide within rat liver after intravenous administration. J Pharmacol Exp Ther. 286, (1), 447-458.
43. Sahashi, K., et al., (2013). Pathological impact of SMN2 mis-splicing in adult SMA mice. EMBO Mol Med. 5, (10), 1586-1601.
44. Olivieri, J. E., et al., (2021). RNA splicing programs define tissue compartments and cell types at single-cell resolution. Elife. 10.
45. Choi, S., et al., (2022). RBFOX2-regulated TEAD1 alternative splicing plays a pivotal role in Hippo-YAP signaling. Nucleic Acids Res. 50, (15), 8658-8673.
46. Xing, S., et al., (2019). DIS3L2 Promotes Progression of Hepatocellular Carcinoma via hnRNP U-Mediated Alternative Splicing. Cancer Res. 79, (19), 4923-4936.
47. Vu, N. T., et al., (2013). hnRNP U enhances caspase-9 splicing and is modulated by AKT-dependent phosphorylation of hnRNP L. J Biol Chem. 288, (12), 8575-8584.
48. Wurster, C. D.& A. C. Ludolph, (2018). Nusinersen for spinal muscular atrophy. Ther Adv Neurol Disord. 11, 1756285618754459.
49. Passini, M. A., et al., (2011). Antisense oligonucleotides delivered to the mouse CNS ameliorate symptoms of severe spinal muscular atrophy. Sci Transl Med. 3, (72), 72ra18.
50. Liu, F., et al., (2015). Mechanosignaling through YAP and TAZ drives fibroblast activation and fibrosis. Am J Physiol Lung Cell Mol Physiol. 308, (4), L344-357.
51. Wang, X., et al., (2020). Cholesterol Stabilizes TAZ in Hepatocytes to Promote Experimental Non-alcoholic Steatohepatitis. Cell Metab. 31, (5), 969-986 e967.
52. Urushima, H., et al., (2021). Activation of Hepatic Stellate Cells Requires Dissociation of E-Cadherin-Containing Adherens Junctions with Hepatocytes. Am J Pathol. 191, (3), 438-453.
53. Marra, F., et al., (2000). Ligands of peroxisome proliferator-activated receptor gamma modulate profibrogenic and proinflammatory actions in hepatic stellate cells. Gastroenterology. 119, (2), 466-478.
54. Fu, M., et al., (2001). Peroxisome proliferator-activated receptor gamma inhibits transforming growth factor beta-induced connective tissue growth factor expression in human aortic smooth muscle cells by interfering with Smad3. J Biol Chem. 276, (49), 45888-45894.
55. Jeon, K. I., R. P. Phipps, P. J. Sime, & K. R. Huxlin, (2015). Inhibitory effects of PPARgamma ligands on TGF-beta1-induced CTGF expression in cat corneal fibroblasts. Exp Eye Res. 138, 52-58.
56. El Ouarrat, D., et al., (2020). TAZ Is a Negative Regulator of PPARgamma Activity in Adipocytes and TAZ Deletion Improves Insulin Sensitivity and Glucose Tolerance. Cell Metab. 31, (1), 162-173 e165.
57. Younossi, Z., et al., (2019). Nonalcoholic Steatohepatitis Is the Fastest Growing Cause of Hepatocellular Carcinoma in Liver Transplant Candidates.

Clin Gastroenterol Hepatol. 17, (4), 748-755 e743.
58. Li, P., et al., (2016). Hematopoietic-Derived Galectin-3 Causes Cellular and Systemic Insulin Resistance. Cell. 167, (4), 973-984 e912.
59. Boigk, G., L. Stroedter, H. Herbst, J. Waldschmidt, E. O. Riecken, & D.

Schuppan, (1997). Silymarin retards collagen accumulation in early and advanced biliary fibrosis secondary to complete bile duct obliteration in rats. Hepatology. 26, (3), 643-649.
60. Chien, W., et al., (2011). Expression of connective tissue growth factor (CTGF/CCN2) in breast cancer cells is associated with increased migration and angiogenesis. Int J Oncol. 38, (6), 1741-1747.

1. A method to prevent, inhibit or treat liver disease in a mammal, comprising: administering to the mammal a composition comprising an effective amount of an antisense oligonucleotide specific for human Hippo pathway transcription factor, TEAD1.

2. The method of claim 1 wherein the mammal is a human.

3.-5. (canceled)

6. The method of claim 1, wherein the antisense oligonucleotide has at least 80%, 82%, 84%, 85%, 87%, 90°/%, 92%, 94%, 95%, 97%, 98% or 99% nucleotide sequence identity to ASO 56.

7. The method of claim 1, wherein the antisense oligonucleotide has at least one or more nucleotide analogs.

8. The method of claim 1, wherein the mammal has or is at risk of having liver fibrosis

9. The method of claim 1, wherein the mammal has or is at risk of having Primary Sclerosing Cholangitis (PSC) or Non-alcoholic steatohepatitis (NASH).

10. A method to prevent, inhibit or treat fibrosis in a mammal, comprising: administering to the mammal a composition comprising an effective amount of an antisense oligonucleotide specific for human Hippo pathway transcription factor, TEAD1.

11. The method of claim 10 wherein the mammal is a human.

12.-15. (canceled)

16. The method of claim 10, wherein the antisense oligonucleotide has at least 80%, 82%, 84%, 85%, 87%, 90%, 92%, 94%, 95%, 97%, 98% or 99% nucleotide sequence identity to ASO 56.

17. The method of claim 10, wherein the antisense oligonucleotide has at least one or more nucleotide analogs.

18. The method of claim 10, wherein the mammal has or is at risk of having liver fibrosis.

19. The method of claim 1, wherein the composition comprises liposomes.

20. The method of claim 1, wherein the composition comprises nanoparticles.

21. A method to prevent, inhibit or treat cholangitis in a mammal, comprising: administering to the mammal a composition comprising an effective amount of an antisense oligonucleotide specific for human Hippo pathway transcription factor, TEAD1.

22.-26. (canceled)

27. The method of claim 21, wherein the antisense oligonucleotide has at least 80%, 82%, 84%, 85%, 87%, 90°/%, 92%, 94%, 95%, 97%, 98% or 99% nucleotide sequence identity to ASO 56.

28. The method of claim 21, wherein the antisense oligonucleotide has at least one or more nucleotide analogs.

29. The method of claim 21, wherein the composition comprises liposomes.

30. The method of claim 21, wherein the composition comprises nanoparticles.

31. A composition comprising an antisense oligonucleotide specific for human Hippo pathway transcription factor, TEAD1.

32. The composition of claim 31 wherein the wherein the antisense oligonucleotide has at least 80%, 82%, 84%, 85%, 87%, 90%, 92%, 94%, 95%, 97%, 98% or 99% nucleotide sequence identity to ASO 56.
